(19) United States
(12) Patent Application Publication
(10) Pub. No.: US 2026/0345550 A1
(43) Pub. Date: Nov. 5, 2026

(54) CYCLOTRIAZADISULFONAMIDE (CADA) COMPOSITIONS AND METHODS FOR USING THEM TO INHIBIT VIRAL REPLICATION

(71) Applicant: Board of Regents of Nevada System of Higher Education, o/b/o/ the University of Nevada, Reno, Reno, NV (US)

(72) Inventors: Thomas Bell, Reno, NV (US); Subhash Verma, Reno, NV (US); Timsy Uppal, Reno, NV (US); Amarawan Intasiri, Reno, NV (US); Topprasad Kapri, Reno, NV (US); Kabita Adhikari, Reno, NV (US)

(21) Appl. No.: 19/659,839

(22) Filed: Apr. 27, 2026

Related U.S. Application Data
(60) Provisional application No. 63/794,559, filed on Apr. 25, 2025.

(51) Int. Cl. A61K 31/395; A61K 31/506; A61P 31/14; C07D 255/02

(52) U.S. Cl. CPC A61K 31/395; A61K 31/506; A61P 31/14; C07D 255/02

(57) ABSTRACT

Disclosed herein are compositions and methods that include a compound or a hydrate, solvate, geometric isomer or salt thereof, where the compound has a chemical structure:

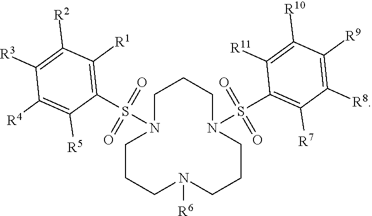

In one specific embodiment, a method of treating a disease includes administering to a subject a therapeutically effective amound of a compound or pharmaceutically acceptable salt of the compound with the above chemical structure.

### CROSS-REFERENCE TO RELATED APPLICATION

This application claims the benefit of U.S. Provisional Patent Application No. 63/794,559, filed Apr. 25, 2025, which is incorporated by reference herein in its entirety.

### STATEMENT REGARDING FEDERALLY SPONSORED RESEARCH OR DEVELOPMENT

This invention was made with Government support under Agreement No. GRANT-1R21AI154254-01, awarded by the National Institute of Allergy and Infectious Diseases (NIAID) a division of the National Institutes of Health (NIH). The United States Government has certain rights in the invention.

### BACKGROUND

### Field

Cyclotriazadisulfonamide compositions and methods for using them in therapeutic compositions to inhibit viral transcription and replication to treat diseases.

### Description of Related Art

The COVID-19 pandemic, driven by the highly infectious severe acute respiratory syndrome coronavirus-2 (SARS-CoV-2), remains a major global health concern despite the availability of vaccines. This underscores the critical need for effective therapies to complement vaccination efforts. While existing antiviral treatments primarily target the main protease (Mpro), developing drugs against additional viral targets, such as the RNA-dependent RNA polymerase (RdRp) to inhibit viral transcription and replication could be an attractive strategy for designing novel antiviral drugs, as it is a broad-spectrum antiviral target for coronaviruses. In this study, a cell-based assay was used to determine the enzymatic activity of SARS-CoV-2 RdRp through luciferase reporter. Various CADA compounds were assessed for their ability to inhibit the enzymatic activity of RdRp, among which TK018 effectively inhibited SARS-CoV-2 RdRp activity with minimal cytotoxicity. The inhibitory effect of TK018 was further evaluated on SARS-CoV-2 replication in cell-based infection assay by determining the levels of infectious virions following treatment of infected cells. Broad spectrum anti-viral activity of these compounds was assessed by treating the cells infected with other human coronaviruses (HCoVs). Using representatives of alpha coronavirus, 229E (HCoV-229E), and OC43 beta-coronavirus (HCoV-OC43) through a cells-based infection system, TK018 was determined to inhibit the replication of tested viruses. The findings highlight the potential of TK018 as a lead compound for developing therapeutic/prophylactic approaches to combat SARS-CoV-2 and future coronaviruses, improving pandemic response and treatment outcomes.

Severe acute respiratory syndrome coronavirus 2 (SARS-CoV-2), which caused the life threating coronavirus disease 2019 (COVID-19), is a novel coronavirus first identified in Wuhan, China, in 2019 (1-3). Since then, the virus has spread globally, causing a worldwide pandemic (4, 5). As reported by the World Health Organization (WHO), as of September 2024, there have been more than 776 million reported cases and over 7 million deaths worldwide. SARS-CoV-2 is a highly contagious virus from the beta-coronavirus genus (1). SARS-CoV-2 is an enveloped virus with a positive-sense single-stranded RNA genome (6). The SARS-CoV-2 virus spreads through respiratory droplets when an infected person talks, coughs, or sneezes (7). Most individuals develop mild to severe symptoms, but immunocompromised patients are more prone to severe illness(6). In addition to SARS-CoV-2, other human coronaviruses OC43 and 229E are known to infect humans. OC43, a Betacoronavirus, and 229E, an Alphacoronavirus, typically cause mild to moderate upper respiratory tract infections, which resemble the common cold and often present with symptoms like nasal congestion, sore throat, and coughing (8). The clinical outcomes of these viruses can be severe in individuals with compromised immune systems, pre-existing health conditions, or elderly populations (9, 10)

The original SARS-CoV-2 B.1 strain underwent rapid genetic mutations, giving rise to several variants of concern (VOCs) characterized by increased transmissibility and severity of infection (7). The World Health Organization (WHO) has classified the primary VOCs as Alpha (B.1.1.7), Beta (B.1.351), Gamma (P.1), Delta (B.1.617.2), and Omicron (BA.1.1.529) (11). Among these, the Omicron variant emerged as a particularly dynamic variant undergoing significant mutations in its spike proteins by early 2022, leading to multiple sub-variants with closely related mutation patterns (12). The Omicron subvariants BA.1, BA.2, BA.4, BA.5, BA.2.75, BQ.1, XBB, XBB.1.5, and JN.1 exhibit different rates of transmissibility and pathogenicity (WHO & (12). Despite the advancements in vaccines and therapeutic strategies, the evolution of new variants poses a daunting challenge to developing effective treatments. The major concerning feature among new variants is the ability to escape immune response, diminishing the effectiveness of the existing vaccination and treatment regimens. This will increase the likelihood of increased infection and severity, particularly among high-risk populations, emphasizing the need for developing newer treatment strategies (7).

The FDA has authorized several antiviral treatments for managing mild to moderate COVID-19 in specific patient populations. Remdesivir, developed by Gilead Sciences, is approved for hospitalized and non-hospitalized individuals at high risk of progressing to severe disease. Molnupiravir, developed by Merck, is authorized under an Emergency Use Authorization (EUA) for adults at high risk for severe outcomes who lack access to or are not eligible for other treatment options. Similarly, Paxlovid, developed by Pfizer, is authorized under an EUA for patients aged 12 years and older (weighing at least 40 kg) who are at high risk of severe illness, including hospitalization or death (13). These treatments play a critical role in managing the disease, especially in vulnerable populations, and reflect ongoing efforts to mitigate the impact of COVID-19 (14). These antivirals have effectively inhibited the replication of SARS-CoV, MERS, and SARS-CoV-2 in both in vitro and in vivo models (3, 15). Remdesivir is a nucleotide analog prodrug that inhibits RNA-dependent RNA polymerase, a critical enzyme for viral replication (16). Similarly, Molnupiravir, a nucleoside analog, acts by inducing lethal mutagenesis in the viral RNA during replication, effectively disrupting the integrity of the viral genome (17, 18). In contrast, Paxlovid functions as a protease inhibitor, blocking polypeptide cleavage and inhibiting the maturation of viral proteins necessary to assemble new viral particles (19-21). Although developing these antivirals represents significant progress, the need for a broad-spectrum antiviral that explicitly targets the inhibition of viral replication, particularly the RNA-dependent RNA polymerase (RdRP), remains critical. Such a broad-spectrum antiviral would be effective against current human coronaviruses and provide preparedness for future outbreaks. Furthermore, it would serve as a vital treatment option for protecting high-risk individuals, offering a more versatile and comprehensive approach to managing coronavirus infections.

Consequently, there is a need for new compositions and methods that can be used to suppress RNA-dependent RNA polymerase activity to inhibit viral transcription and replication to treat diseases, such as COVID-19.

### SUMMARY

Provided herein are compositions and methods for therapeutic compositions. In one specific embodiment, a compound or a hydrate, solvate, geometric isomer or salt thereof, where the compound has a chemical structure:

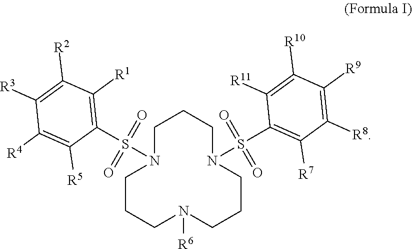

In another specific embodiment, a method of treating a disease including administering to a subject a therapeutically effective amound of a compound or pharmaceutically acceptable salt of a compound with a chemical structure of Formula I.

### BRIEF DESCRIPTION OF THE DRAWINGS

For the purpose of promoting an understanding of the principles of the present disclosure, reference is now made to the embodiments illustrated in the drawings, which are described below. The embodiments disclosed herein are not intended to be exhaustive or limit the present disclosure to the precise form disclosed in the following detailed description. Rather, the embodiments are chosen and described so that others skilled in the art can utilize their teachings. Therefore, no limitation of the scope of the present disclosure is thereby intended.

FIG. 1 is a graph showing the screening of the CADA compounds was performed using a HEK293L cell-based reporter system. Firefly (FLuc) and Renilla (RLuc) luciferase values were plotted, and the FLuc/RLuc ratio (index values), an indicator of RdRp inhibition.

FIG. 2 is an immunoblot for SARS-CoV-2 NSP12, NSP8, and NSP7 proteins detected using anti-SARS-CoV-2 NSP12, NSP8, NSP7, and GAPDH antibodies following 48 h of treatment.

FIG. 3 is a graph of the RdRp inhibitory concentrations of TK018 at 1.5625, 3.125, 6.25, 12.5, 25, 50, and 100 μM carried out following 48 hours of treatment. FLuc and RLuc levels were measured, and the FLuc/RLuc ratio index was determined.

FIG. 4 is a graph showing the non-linear regression analysis to determine IC50 value.

FIG. 5 is a graph showing the cytotoxicity analysis of TK018 in 293 L and Vero-TMPRSS2 cells through MTT assay. The toxicity of TK018 at 1.5625, 3.125, 6.25, 12.5, 25, 50, and 100 μM concentrations on HEK293 cells was analyzed by MTT assay, 48 hours post-treatment.

FIG. 6 is a graph showing the 50% cytotoxic concentration of TK018 on HEK293 cells.

FIG. 7 is a graph showing the cytotoxicity analysis of TK018 at 1.5625, 3.125, 6.25, 12.5, 25, 50, and 100 μM concentrations on Vero-TMPRSS2 cells was analyzed by MTT assay, 48 hours post-treatment.

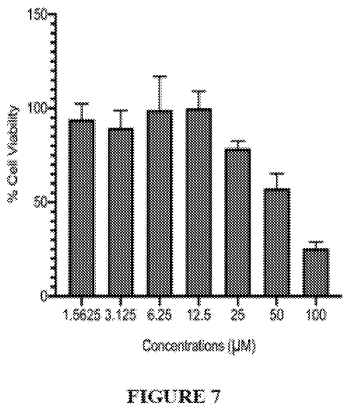

FIG. 8 is a graph showing the 50% cytotoxic concentration of TK018 on Vero-TMPRSS2 cells.

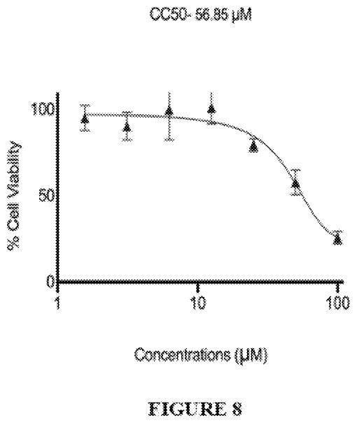

FIG. 9 is a graph showing the screening of the CADA compounds for Vero-TMPRSS2 cells infected with USA-WA1/2020 treated with 10 μM of the CADA compound or DMSO (control) for 48 h. RNA extracted from the infected and treated cells was used to quantify the intracellular SARS-CoV-2 genome by rt-qPCR.

FIG. 10 shows the infectious viral particles in the supernatants quantified in a plaque assay by infecting the Vero-TMPRSS2 cell monolayers. Varying amounts of supernatants (50 μL, 5 μL, 0.5 μL, and 0.05 μL) were used for determining the plaque-forming units. After 3 days of CMC-MEM overlay, the cell monolayers were fixed and stained with crystal violet to visualize and count the plaque-forming units (PFU/mL).

FIG. 11 is a graph showing the reduction in intracellular viral genome copies in infected cells treated with varying concentrations of TK018.

FIG. 12 is a graph showing the IC50 value of TK018 against USA-WA1/2020 was determined by measuring the reduction in intracellular viral genome copies in infected cells treated with varying concentrations of TK018.

FIG. 13 is a graph showing the screening of the CADA compounds for Vero-TMPRSS2 cells infected with HCoV-OC43 and treated with DMSO (control) or 10 μM of the CADA compounds for 48 h. RNA extracted from the infected and treated cells and supernatants were used to quantify the HCoV-OC43 RNA genome by rt-qPCR.

FIG. 14 is an immunoblot using HCoV-OC43 nucleocapsid and GAPDH antibodies following 48 h of treatment to detect the nucleocapsid protein and loading control GAPDH expression.

FIG. 15 shows the determination of infectious virus particles in the infected and compound-treated supernatants performed through a plaque assay. Supernatant volumes of 500 μL, 50 μL, and 5 μL from HCoV-OC43-infected cells and DMSO-treated or CADA-compound-treated supernatants were used for the plaque assay. Plaques were counted to determine plaque-forming units per mL (PFU/mL), with nc indicating not countable.

FIG. 16 is a graph is showing the reduction in intracellular viral genome copies in infected cells treated with varying concentrations of TK018.

FIG. 17 is a graph showing the IC50 value of TK018 against HCoV-OC43 determined by measuring the reduction in intracellular viral genome copies in infected cells treated with varying concentrations of TK018.

FIG. 18 is a graph showing the screening of the CADA compounds for Vero-TMPRSS2 cells were infected with HCoV-229E and treated with DMSO (control) or 10 μM of the CADA compounds for 48 h. RNA extracted from the infected and treated cells and supernatants were used to quantify the HCoV-229E RNA genome by rt-qPCR.

FIG. 19 is an immunoblot for HCoV-229E nucleocapsid protein and loading control GAPDH expression using HCoV-229E nucleocapsid and GAPDH antibodies following 48 h post-treatment.

FIG. 20 shows the determination of infectious virus particles in the infected and compound-treated supernatants was performed through a plaque assay. Supernatant volumes of 500 μL, 50 μL, and 5 μL from HCoV-229E-infected cells and DMSO-treated or CADA-compound-treated supernatants were used for the plaque assay. Plaques were counted to determine plaque-forming units per mL (PFU/mL), with nc indicating not countable.

### DETAILED DESCRIPTION

The antiviral properties of selected analogs of cyclotriazadisulfonamide (CADA) compounds and Nilotinib were studied. Some CADA analogs were found to decrease the expression of the cell-surface cluster of differentiation 4 (CD4) protein on primate cells and inhibit replication of human immunodeficiency virus (HIV) (22, 23). CD4 is required for the entry of HIV into host cells (22). Hence, these CADA compounds act as HIV entry inhibitors (24). It has been found that the lead compound (CADA) down-modulates cell-surface CD4 by binding the signal peptide (SP) of the CD4 preprotein (25, 26). CADA binds to the SP of the human CD4 preprotein, preventing co-translational translocation of CD4 into the endoplasmic reticulum (ER) lumen, significantly reducing its expression on the cell surface (26). SP-targeting compounds could potentially be developed as drugs to inhibit the expression of many cell-surface proteins of medicinal interest. For example, CADA also down-modulates another human protein, Sortilin, as a secondary target (27). Sortilin has been identified as a potential drug target for various disorders, including frontotemporal lobar degeneration, autism, Alzheimer's disease, atherosclerosis, and breast cancer (28-31). Preliminary studies suggest that different analogs of CADA compounds may function as a broad antiviral agent by downregulating signal peptides of transmembrane proteins involved in viral replication or entry like angiotensin-converting enzyme 2-ACE2 (SARS-CoV and SARS-CoV-2), hemagglutinin (H1N1 influenza), MARTX protein (cholera), plasmepsin X (malaria) among others (32). During the compound library screening, results with Nilotinib were noteworthy. This commercially available tyrosine kinase inhibitor has been explored as a treatment for various types of chronic leukemia and was therefore included in the study for further investigation.

A library of CADA compounds to identify inhibitors of RNA-dependent RNA polymerase (RdRp), a critical enzyme for the replication and transcription of SARS-CoV-2, were screened. SARS-CoV-2 RdRp (nsp12) is a key target for antiviral drug development due to its vital role in viral replication and highly conserved sequence homology among HCoVs. Given this conserved homology, targeting RdRp offers the potential for broad-spectrum antiviral strategies, not just for SARS-CoV-2 but also for other HCoVs. In this study, SARS-CoV-2 reporter assay (33) and a live virus infection assay, including SARS-CoV-2 WA1, HCoV-OC43, and HCoV-229E were used to evaluate the antiviral efficacy of the CADA compound library. The findings revealed that the compound TK018 was particularly effective at inhibiting SARS-CoV-2 replication. Furthermore, in live virus assays, TK018 demonstrated broad-spectrum antiviral activity when tested against HCoV-OC43 and HCoV-229E. These results highlight the potential of TK018 as a promising candidate for the development of broad-spectrum antiviral therapies targeting RdRp.

The compositions provided herein can include compounds of Formula I:

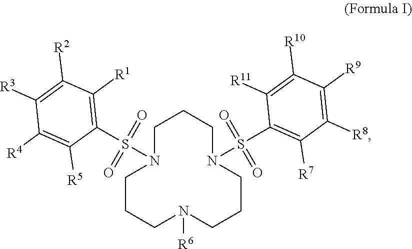

where R¹, R², R³, R⁴, R⁵, R⁶, R⁷, R⁸, R⁹, R¹⁰, and R¹¹ can be independently selected from the group consisting of: H; F; Cl; Br; I; OH; ketone (═O); ether [—OR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; acyl halide (—COX); carbonyl [—COR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; aldehyde (—CHO); carbonate ester [—OCOOR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; carboxyl (—COOH); amide [—CONR′R″, where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; amines [—NR′R″, where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; cyanate (—OCN); isocyanate (—NCO); nitrate (—ONO₂); nitrile (—CN); isonitrile (—NC); nitroso (—NO); oxime (—CH═NOH); borono —B(OH)₂; boronare [—B(OR′XR″), where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; borinate [—B(R′)(OR″), where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; phosophino [—PR₂, where R can include hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; phosphono [—P(═O)(OH)(R), where R can include hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; phosphate (—OP(═O)(OH)₂; thiol (—SH); sulfide [—SR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; disulfide [—SSR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers], sulfinyl [—S(═O)R, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; sulfino (—SO₂H); sulfo (—SO₃H); thiocyanate; isothiocyanate; carbonothioyl [—C(═S)R where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; (C₁₋₄)alkyl, such as methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers; and where X can be independently selected from the group consisting of: CH, S, NH, and O.

The compounds of Formula I can include:

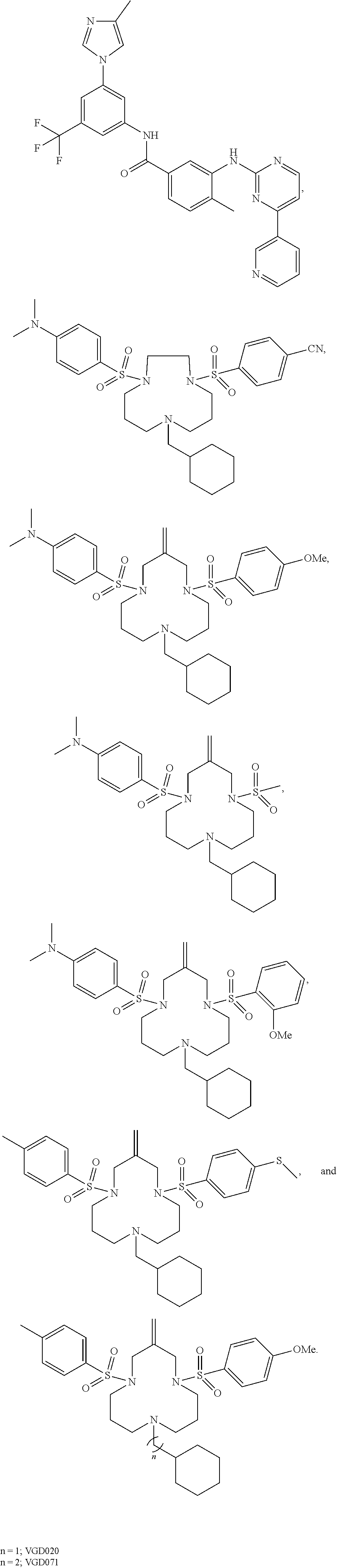

It has been discovered that compounds of Formula I can inhibit viral transcription and replication for the treatment of diseases. The compounds of Formula I can be provided in many forms. For example, the compounds of Formula I can include, but are not limited to, salts, hydrates, solvates, isomers, crystalline and non-crystalline forms, isomorphs, polymorphs, and metabolites thereof. The compounds of Formula I may exist in unsolvated and solvated forms. For example, when the solvent, such as water, is tightly bound, the complex will have a well-defined stoichiometry independent of humidity. When, however, the water is weakly bound, as in channel solvates and hygroscopic compounds, the water/solvent content can depend on humidity and drying conditions; hence, non-stoichiometry will be the norm. Moreover, the compounds of Formula I may exist in unsolvated as well as solvated forms with pharmaceutically acceptable solvents, such as water and ethanol. In general, the solvated forms are considered equivalent to the unsolvated forms. The compounds of Formula I may also exist in one or more crystalline states, i.e., polymorphs, or they may exist as amorphous solids.

Many of the compounds of Formula I may exist as geometric isomers or possess one or more asymmetric centers, thus existing as two or more stereoisomeric forms. The compounds of Formula I can include all the individual stereoisomers and geometric isomers of the compounds and mixtures thereof. Individual enantiomers can be obtained by chiral separation or using the relevant enantiomer in the synthesis.

The compounds of Formula I can include asymmetric carbons. When present in racemic compounds, solid and dotted wedges are used to define relative stereochemistry, rather than absolute stereochemistry. Racemic compounds possessing such indicated relative stereochemistry are marked with (+/−). Unless stated otherwise, it is intended that the compounds of Formula I can exist as stereoisomers, which include cis and trans isomers, optical isomers such as R and S enantiomers, diastereomers, geometric isomers, rotational isomers, conformational isomers, atropisomers, and mixtures thereof (such as racemates and diastereomeric pairs). The compounds of Formula I may exhibit more than one type of isomerism. Also included are acid addition or base addition salts wherein the counterion is optically active, for example, D-lactate or L-lysine, or racemic, for example, DL-tartrate or DL-arginine. When any racemate crystallizes, crystals of two different types are possible. The first type is the racemic compound (true racemate) where one homogeneous form of crystal is produced containing both enantiomers in equimolar amounts. The second type is the racemic mixture or conglomerate wherein two forms of crystal are produced in equimolar amounts each comprising a single enantiomer.

The compounds of Formula I can also include prodrugs. Thus, certain compounds of Formula I that may have little or no pharmacological activity themselves can, when administered into or onto the body, be converted into a compound having the desired activity, for example, by hydrolytic cleavage. Further information on the use of prodrugs may be found in “Pro-drugs as Novel Delivery Systems, Vol. 14, ACS Symposium Series (T. Higuchi and W. Stella) and “Bioreversible Carriers in Drug Design,” Pergamon Press, 1987 (ed. E. B. Roche, American Pharmaceutical Association). Prodrugs can, for example, be produced by replacing appropriate functionalities present in the compounds of Formula I with certain moieties known to those skilled in the art as “pro-moieties” as described, for example, in “Design of Prodrugs” by H. Bundgaard (Elsevier, 1985).

The compounds of Formula I can include all pharmaceutically acceptable isotopically labeled compounds, which are identical to those recited herein, wherein one or more atoms are replaced by an atom having the same atomic number, but an atomic mass or mass number different from the atomic mass or mass number which predominates in nature. Examples of isotopes suitable for inclusion in the compounds of Formula I can include, but are not limited to, isotopes of hydrogen, such as ²H, ³H; carbon, such as ¹¹C, ¹³C, and ¹⁴C; chlorine, such as ³⁶Cl; fluorine, such as ¹⁸F; iodine, such as ¹²³I and ¹²⁵I; nitrogen, such as ¹³N and ¹⁵N; oxygen, such as ¹⁵O, ¹⁷O, and ¹⁸O; phosphorus, such as ³²P; and sulfur, such as ³⁵S. Certain isotopically-labeled compounds of Formula I, for example, those incorporating a radioactive isotope, are useful in drug and/or substrate tissue distribution studies (e.g., assays). The radioactive isotopes tritium, i.e., ³H, and carbon-14, i.e., ¹⁴C, are particularly useful for this purpose in view of their ease of incorporation and ready means of detection. Substitution with heavier isotopes such as deuterium, i.e., ²H, may afford certain therapeutic advantages resulting from greater metabolic stability, for example, increased in vivo half-life or reduced dosage requirements and, hence, may be preferred in some circumstances. Substitution with positron-emitting isotopes, such as ¹¹C, ¹⁵F, ¹⁸F, ¹⁵O and ³N, can be useful in positron emission tomography studies for examining substrate receptor occupancy. Isotopically labeled compounds of Formula I can generally be prepared by conventional techniques or by processes analogous to those described in Scheme 1 or the Examples (see below) using an appropriate isotopically labeled reagent in place of the non-labeled reagent previously employed. Pharmaceutically acceptable solvates can include those wherein the solvent of crystallization may be isotopically substituted, e.g., D₂O, acetone-d₆, or DMSO-d₆. Compounds of Formula I can include, but are not limited to, isotopically labeled versions of these compounds, such as, but not limited to, the deuterated and tritiated isotopes and all other isotopes discussed above.

The compounds of Formula I can be provided in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

When a salt is intended to be administered to a subject the salt can be provided in a pharmaceutically acceptable form. A pharmaceutically salt can prepared by combining a compound of Formula I with an acid whose anion, or a base whose cation, is generally considered suitable for mammalian consumption. Pharmaceutically acceptable salts of the compounds of Formula I can have greater aqueous solubility relative to the parent compound.

Suitable pharmaceutically salts of the compounds of Formula I can include those derived from inorganic acids. The inorganic acids can include, but are not limited to, hydrochloric, hydrobromic, hydrofluoric, boric, fluoroboric, phosphoric, meta-phosphoric, nitric, carbonic, sulfonic, and sulfuric acids.

Suitable pharmaceutically salts of the compounds of Formula I can include those derived from organic acids. The organic acids can include, but are not limited to, aliphatic, cycloaliphatic, aromatic, araliphatic, heterocyclic, carboxylic, and sulfonic classes of organic acids. Specific examples of suitable organic acids can include, but are not limited to: acetate, trifluoroacetate, formate, propionate, succinate, glycolate, gluconate, digluconate, lactate, malate, tartrate, citrate, ascorbate, glucuronate, maleate, fumarate, pyruvate, aspartate, glutamate, benzoate, anthranilate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylamino-ethansulfonate, algenic acid, p-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalene-sulfonate, oxalate, palmoate, pectinate, 3-phenylpropionate, picrate, pivalate, thiocyanate, and undecanoate.

Organic salts of the compounds of Formula I may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), and arylalkyl halides (e.g., benzyl and phenethyl bromides).

Furthermore, when the compounds of Formula I carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

The compounds of Formula I or their pharmaceutically acceptable salts, I can be made using many synthetic strategies and known chemical transformations. The reaction Scheme 1, together with synthetic methods known in the art of organic chemistry, or modifications and derivatizations that are familiar to those of ordinary skill in the art, illustrate a method for preparing the compounds of Formula I. The starting materials used can be commercially available or may be prepared by routine methods known in the art. For example, the starting materials can be prepared by the methods discussed and described in the COMPENDIUM OF ORGANIC SYNTHETIC METHODS, Vol. I-XII (published by Wiley-Interscience).

The compounds of Formula I can be made using various Pd catalysts, such as in a Heck reaction. Scheme 1 depicts a Heck reaction that can be used to make compounds of Formula I.

Furthermore, the R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, and R²¹ substituents and any derivatives of the compounds of Formula I can be made by known chemical transformations of the compounds' functional groups using standard chemical reactions. For example, these standard chemical reactions can include, but are not limited to: polar reactions under basic conditions, polar reactions under acidic conditions, pericyclic reactions, and free radical reactions. In another example, these standard chemical reactions can include, but are not limited to: addition reactions, substitution reactions, oxidation reactions, reduction reactions, elimination reactions, hydrolysis, acylation, amidations, etherification, and esterification. Alkane functional group transformations can include, but are not limited to: free radical chlorination (hv, Cl₂), free radical bromination (hv, Br₂), and allylic bromination (NBS). Alkene functional group transformations can include, but are not limited to: addition of HCl, addition of HBr, addition of HI, addition of H₃O⁺, chlorination (Cl₂) bromination (Br₂), iodination (I₂), chlorohydrin formation (Cl₂/H₂O), bromohydrin formation (Br₂/H₂O), ether formation (H⁺/ROH), oxymercuration (Hg(OAc)₂/H₂O), oxymercuration, (Hg(OAc)₂/ROH), hydroboration, epoxidation (RCO₃H), dihydroxylation (OSO₄), dihydroxylation (KMnO₄), cyclopropanation, dichlorocyclopropanation, ozonolysis (reductive workup), ozonolysis (oxidative workup), oxidative cleavage (KMnO₄), hydrogenation, rearrangements (H shift), rearrangements (alkyl shift), free radical addition of HBr, and Sharpless epoxidation. Alkyne functional group transformations can include, but are not limited to: deprotonation (acetylide formation), S_{N}2 with alkyl halides, partial reduction (Lindlar), partial, reduction (Na/NH₃), hydroboration, oxymercuration, addition of HCl, HBr, or HI, addition of HCl, HBr, or HI, hydrogenation, ozonolysis, oxidative cleavage (KMnO₄), and halogenation (Cl₂, Br₂, I₂). The substitution reaction can include, but is not limited to: alcohol formation, nitrile formation, thiol formation, ether formation, thioether formation, azides, ester formation, acetylide addition, alkanes (Gilman reagents), ammonium salt formation, alkyl chloride formation, alkyl bromide formation, alkyl iodide formation, alkyl shift, and hydride shift. Elimination reactions can include, but are not limited to: alkenes from alkyl halides, alkenes from alcohols (strong acid), alkenes from alcohols (POCl₃), alkenes from alkyl halides, E1 with rearrangement (alkyl shift), Hoffmann elimination, and alkyne formation via elimination E1 with rearrangement (hydride shift). Organometallic reactions can include, but are not limited to: Grignard formation (alkyl halides), Grignard formation (alkenyl halides), reaction of Grignards with acids, addition of Grignards to aldehydes, addition of Grignards to ketones, addition of Grignards to esters, reaction of Grignards with CO₂, addition of Grignards to nitriles, formation of organolithium reagents, formation of Gilman reagents, S_{N}2 with Gilman reagents, addition of Gilman reagents to enones, addition of Gilman to acyl halides, Heck reaction, Suzuki reaction, and Stille reaction. Reactions of epoxides can include, but are not limited to: epoxide opening (basic conditions), epoxide opening (acidic conditions), epoxide opening (diol formation), epoxide formation (from halohydrins), epoxide formation (from alkenes), and Sharpless epoxidation of alkenes. Reactions of alcohols and thiols can include, but are not limited to: deprotonation (alkoxide formation), protonation (onium ion formation), conversion to tosylates/mesylates, conversion to alkyl chlorides (SOCl₂), conversion to alkyl bromides (PBr₃), oxidation to aldehydes (PCC), oxidation to ketones (PCC+others), oxidation to carboxylic acid, (H₂CrO₄+ others), protection as silyl ethers, thiol formation (S_{N}2), and thiol oxidation to disulfides. Reactions of dienes can include, but are not limited to: Diels-alder reaction, polymerization of dienes, reactions of aromatics (arenes), nitration (HNO₃/H₂SO₄), chlorination (Cl₂ plus catalyst), bromination (Br₂ plus catalyst), sulfonylation (SO₃/H₂SO₄), Friedel Crafts alkylation (R—X plus catalyst), Friedel Crafts acylation (RCOX plus catalyst), iodination (I₂/catalyst), side chain oxidation (KMnO₄), reduction of nitro groups, reduction of aromatic ketones, side chain bromination, nucleophilic aromatic substitution (SNAr), and aryne formation (S_{N}Ar via arynes). Reactions of aldehydes and ketones can include, but are not limited to: hydrate formation (H₂O), cyanohydrin formation (CN), reduction of aldehydes (NaBH₄), reduction of aldehydes (LiAlH₄), reduction of ketones (NaBH₄), reduction of ketones (LiAlH₄), Grignard addition to aldehydes, Grignard addition to ketones, acetal formation (ROH/H⁺), acetal hydrolysis (H₃O⁺), imine, formation (RNH₂), enamine formation (R₂NH), Wolff-Kishner: reduction to alkanes, Clemmensen, reduction to alkanes, oxidation to carboxylic acid (H₂CrO₄ or KMnO₄), keto-enol tautomerism, enolate formation, aldol addition reaction, alkylation of enolates, Wittig reaction (alkene formation), thioacetal formation, imine hydrolysis, oxidation to carboxylic acids (Tollens), haloform reaction, Baeyer-Villiger reaction, aldol condensation, Cannizarro reaction. Reactions of carboxylic acids can include, but are not limited to: deprotonation (carboxylate formation), formation via Grignard and CO₂, conversion to acid chloride (SOCl₂), reduction (LiAlH₄), Fischer esterification, and decarboxylation (of β-keto acids). Reactions of esters can include, but are not limited to: reduction to aldehydes (DIBAL-H), reduction to alcohols (LiAlH₄), hydrolysis to carboxylic acid (acidic), hydrolysis to carboxylic acid (basic), addition of Grignard reagents to esters, Claisen condensation, and transesterification (basic conditions). Reactions of acyl halides can include, but are not limited to: conversion to esters (ROH), conversion to carboxylic acids (H₂O), conversion to anhydrides (RCO₂), conversion to amides (RNH₂), conversion to ketones (Gilman reagents), and conversion to aldehydes (LiAlH(OtBu)₃). Reactions of α,β-unsaturated ketones (enones) can include, but are not limited to: Michael reaction (conjugate addition of enolates), conjugate addition of Gilman reagents, conjugate addition of other nucleophiles. Reactions of amines and amides can include, but are not limited to: dehydration of amides to nitriles (P₂O₅), Hofmann rearrangement, Gabriel synthesis of amines, reductive amination, formation of diazonium salts, reactions of diazonium salts, amide formation using DCC, amide formation from acid halides, and Curtius rearrangement. Reactions of nitriles can include, but are not limited to: addition of Grignard reagents to nitriles, reduction to amines (LiAlH₄), and hydrolysis to carboxylic acids. Chemical reactions and reagents that may be utilized to make derivatives and prodrugs of the compounds of Formula I are described in M. B. Smith, March's Advanced Organic Chemistry, 7^{th} edition, 2013.

One skilled in the art will recognize that in some cases, the compounds of Formula I made by various synthetic strategies and chemical transformations will be generated as a mixture of diastereomers and/or enantiomers; these may be separated at various stages of the synthetic scheme using conventional techniques or a combination of such techniques, such as, but not limited to, crystallization, normal-phase chromatography, reversed phase chromatography and chiral chromatography, to afford the single enantiomers of the compounds of Formula I.

During any of the following synthetic sequences, it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991; and T. W. Greene and P. G. M. Wuts, Protective Groups in Organic Chemistry, John Wiley & Sons, 1999; and T. W. Greene and P. G. M. Wuts, Protective Groups in Organic Chemistry, John Wiley & Sons, 2006, which are hereby incorporated by reference.

The diseases that can be treated with compounds of Formula I can include, but are not limited to: idiopathic pulmonary fibrosis, amyotropic lateral sclerosis, Alzheimer's disease, Parkinson's disease, Huntington's disease, and other syncleinopathies and taupathies, such as Parkinson's disease dementia and frontotemporal lobe dementia and other dementia's and memory loss conditions which may be associated with age related increases with neurotoxic protein aggregation and/or increased oxidative stress or to defect of autophagy (the cells mechanism for removing damaged cellular components). The compounds of Formula I can also be used to relieve oxidative stress and promote autophagy.

Suitable subjects that can be treated by compounds of Formula I can include mammalian subjects. Mammals can include, but are not limited to, canine, feline, bovine, caprine, equine, ovine, porcine, rodents, lagomorphs, and primates. In one embodiment, the subjects are humans. Human subjects may be of either gender and at any stage of development.

The compounds of Formula I can be incorporated into pharmaceutical compositions. For example, a pharmaceutical composition can include a mixture of one or more compounds of Formula I and one or more pharmaceutically acceptable carriers. In another example, a pharmaceutical composition can include a mixture of one or more compounds of Formula I, one or more pharmaceutically acceptable carriers, and one or more adjuvants. Other pharmacologically active substances can also be present. The compounds of Formula I can be administered as a compound per se or the compounds of Formula I can be administered as pharmaceutically acceptable salts. Pharmaceutically acceptable salts of compounds of Formula I can have greater aqueous solubility relative to the parent compound.

Compositions that include compounds of Formula I can be formulated according to known methods for preparing pharmaceutically useful compositions. Formulations are described in detail in a number of sources which are well known and readily available to those skilled in the art. For example, Remington's Pharmaceutical Science by E. W. Martin describes how to make pharmaceutical formulations. In general, the compositions that include compounds of Formula I can be formulated such that an effective amount of at least one compound of Formula I is combined with a suitable pharmaceutically acceptable carrier or diluent in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art.

The one or more mixtures can have a content of the one or more compounds of Formula I that varies widely. For example, the mixtures can have a content of the one or more compounds of Formula I from a low of about 0.1 wt %, about 1.0 wt %, or about 5.0 wt %, to a high of about 90.0 wt %, about 95.0 wt %, or about 99.9 wt %. In another example, the mixtures can have a content of the one or more compounds of Formula I from about 0.1 wt % to about 99.9 wt %, about 1.0 wt % to about 99.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. The weight percent of the one or more compounds of Formula I in the mixtures can be based on the total weight of the mixture.

The one or more reaction mixtures can have a solid to liquid ratio that varies widely. For example, the reaction mixtures can have a solid to liquid ratio from a low of about 1:10, about 1:5, about 1:4, about 1:3, about 1:2, about 1:1, about 2:1, about 3:1, about 4:1, about 5:1, or about 10:1. For example, the one or more reaction mixtures can have a solid to liquid ratio of about 4:1 (mg/mL).

In some embodiments, the one or more additives can include, but is not limited to, one or more adjuvants, one or more acids, one or more bases, one or more catalysts, one or more oxidizing agents, one or more reducing agent, and mixtures thereof.

The one or more reaction mixtures can have a content of the one or more additives that varies widely. For example, the reaction mixtures can have a content of the one or more additives from a low of about 0.1 wt %, about 1.0 wt %, or about 5.0 wt %, to a high of about 90.0 wt %, about 95.0 wt %, or about 99.9 wt %. In another example, the reaction mixtures can have a content of the one or more additives from about 0.1 wt % to about 99.9 wt %, about 1.0 wt % to about 99.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt %, to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. The weight percent of the one or more additives in the reaction mixtures can be based on the total weight of the composition.

The one or more solvent mixtures can have a content of the one or more additives that varies widely. For example, the solvent mixtures can have a content of the one or more additives from a low of about 0.1 wt %, about 1.0 wt %, or about 5.0 wt %, to a high of about 90.0 wt %, about 95.0 wt %, or about 99.9 wt %. In another example, the solvent mixtures can have a content of the one or more additives from about 0.1 wt % to about 99.9 wt %, about 1.0 wt % to about 99.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. The weight percent of the one or more additives in the solvent mixtures can be based on the total weight of the solvent mixtures.

The one or more solvents and/or carrier fluids for the first reaction mixture, second reaction mixture, and catalyst mixture can include, but are not limited to: aliphatic hydrocarbons, such as hexanes; aromatic hydrocarbons, such as toluene and benzene; water; deionized water; methanol; ethanol; propanol; isopropanol; acetone: acetonitrile; chloroform; diethyl ether; methylene chloride; dimethyl formamide; ethylene glycol; propylene glycol; triethylamine; tetrahydrofuran; and mixtures thereof. The one or more reaction mixtures can have a content of the solvents and/or carrier fluids that varies widely.

The one or more reaction mixtures can have a content of the one or more solvents and/or carrier fluids that varies widely. For example, the reaction mixtures can have a content of the one or more solvents and/or carrier fluids from a low of about 0.1 wt %, about 1.0 wt %, or about 5.0 wt %, to a high of about 90.0 wt %, about 95.0 wt %, or about 99.9 wt %. In another example, the reaction mixtures can have a content of the one or more solvents and/or carrier fluids from about 0.1 wt % to about 99.9 wt %, about 1.0 wt % to about 99.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. The weight percent of the one or more solvents and/or carrier fluids in the reaction mixtures can be based on the total weight of the composition.

The one or more solvent mixtures can have a content of the one or more solvents and/or carrier fluids that varies widely. For example, the solvent mixtures can have a content of the one or more solvents and/or carrier fluids from a low of about 0.1 wt %, about 1.0 wt %, or about 5.0 wt %, to a high of about 90.0 wt %, about 95.0 wt %, or about 99.9 wt %. In another example, the solvent mixtures and/or carrier fluids can have a content of the one or more solvents from about 0.1 wt % to about 99.9 wt %, about 1.0 wt % to about 99.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 w %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. The weight percent of the one or more solvents and/or carrier fluids in the solvent mixtures can be based on the total weight of the solvent mixtures.

In some embodiments, one or more critical metals and/or one or more valuable materials can be recovered from the one or more lithium-ion battery black mass compositions. For example, a critical metal that can be recovered from the lithium-ion battery black mass composition can include, but is not limited to: lithium, cobalt, manganese, nickel, and combinations thereof.

The one or more lithium-ion battery black mass compositions can have a content of the one or more valuable materials that varies widely. For example, the spent lithium-ion battery black mass composition can have a content of the one or more valuable materials from a low of about 0.1 wt %, about 1.0 wt %, or about 5.0 wt %, to a high of about 90.0 wt %, about 95.0 wt %, or about 99.9 wt %. In another example, the spent lithium-ion battery black mass composition can have a content of the one or more valuable materials from about 0.1 wt % to about 99.9 wt %, about 1.0 wt % to about 99.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. The weight percent of the one or more valuable materials in the spent lithium-ion battery black mass compositions can be based on the total weight of the composition.

The one or more acids can include, but are not limited to, hydrochloric acid, sulfuric acid, phosphoric acid, citric acid, nitric acid, humic acid, acetic acid, carbonic acid, formic acid, and combinations thereof. The one or more reaction mixtures can have a content of the one or more acids that varies widely. For example, the reaction mixtures can have a content of the one or more acids from a low of about 0.1 wt %, about 1.0 wt %, or about 5.0 wt %, to a high of about 90.0 wt %, about 95.0 wt %, or about 99.9 wt %. In another example, the reaction mixtures can have a content of the one or more acids from about 0.1 wt % to about 99.9 wt %, about 1.0 wt % to about 99.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. In another example, the one or more acids can include sulfuric acid and phosphoric acid in a ratio of about 12:1, about 10:1, about 9:1, about 8:1 about 7:1, or about 6:1. In another example, the reaction mixtures can be free of the one or more acids. The weight percent of the acid in the reaction mixtures can be based on the total weight of the composition.

The one or more bases can include, but are not limited to, sodium hydroxide, calcium hydroxide, potassium hydroxide, sodium phosphate, and combinations thereof. The one or more reaction mixtures can have a content of the one or more bases that varies widely. For example, the reaction mixtures can have a content of the one or more bases from a low of about 0.1 wt %, about 1.0 wt %, or about 5.0 wt %, to a high of about 90.0 wt %, about 95.0 wt %, or about 99.9 wt %. In another example, the reaction mixtures can have a content of the one or more bases from about 0.1 wt % to about 99.9 wt %, about 1.0 wt % to about 99.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt^{%}, about 50.0 wt % to about 60.0 wt %, or about 70.0 wt % to about 80.0 wt %. In another example, the reaction mixtures can be free of the one or more bases. The weight percent of the base in the reaction mixtures can be based on the total weight of the composition.

The pH of the one or more reaction mixtures can vary widely. For example, the one or more reaction mixtures can have a pH from about 4.0 to about 12.0, about 5.0 to about 10.0, about 7.5 to about 11.0, about 7.0 to about 10.0, about 8.0 to about 9.0, about 9.0 to about 10.0, about 8.0 to about 10.0, about 9.0 to about 11.0, or about 6.0 to about 9.0.

The one or more salts can include, but are not limited to: cesium formate, sodium chloride, sodium carbonate, sodium bicarbonate, potassium chloride, potassium carbonate, potassium bicarbonate, potassium fluoride, sodium fluoride, potassium formate, sodium formate, calcium chloride, ammonium carbonate, ammonium chloride, tetramethylammonium chloride, sodium chloride, potassium chloride, and mixtures thereof. The one or more reaction mixtures can have a content of the one or more salts that varies widely. For example, the reaction mixtures can have a content of the one or more salts from a low of about 0.1 wt %, about 1.0 wt %, or about 5.0 wt %, to a high of about 90.0 wt %, about 95.0 wt %, or about 99.9 wt %. In another example, the reaction mixtures can have a content of the one or more salts from about 0.1 wt % to about 99.9 wt %, about 1.0 wt % to about 99.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. In another example, the reaction mixtures can be free of the one or more salts. The weight percent of the salts in the reaction mixtures can be based on the total weight of the composition.

The one or more solvents and/or carrier fluids of the reaction mixtures can include, but are not limited to: aliphatic hydrocarbons, such as hexanes; aromatic hydrocarbons, such as toluene and benzene; water; brines; seawater; oil and gas-produced water; lithium battery waste solutions; methanol; ethanol; propanol; isopropanol; acetone; acetonitrile; chloroform; diethyl ether; methylene chloride; dimethyl formamide; ethylene glycol; propylene glycol; triethylamine; tetrahydrofuran; and mixtures thereof.

The one or more reaction mixtures can have a content of the solvents and/or carrier fluids that varies widely. For example, the reaction mixtures can have a content of the solvents and/or carrier fluids from a low of about 0.1 wt %, about 0.5 wt %, or about 1.0 wt %, to a high of about 50.0 wt %, about 70.0 wt %, or about 99.9 wt %. In another example, the reaction mixtures can have content of the solvents and/or carrier fluids from about 0.1 wt % to about 99.9 wt %, 0.2 wt % to about 10 wt %, about 0.5 wt % to about 10.0 wt %, about 2.0 wt % to about 20.0 wt %, about 5.0 wt % to about 60.0 wt %, about 15.0 wt % to about 25.0 wt %, about 17.0 wt % to about 54.0 wt %, about 19.0 wt % to about 27.0 wt %, about 15.0 wt % to about 27.0 wt %, about 14.0 wt % to about 24.0 wt %, about 11.0 wt % to about 28.0 wt %, about 33.0 wt % to about 48.0 wt %, about 51.0 wt % to about 54.0 wt %, or about 50.0 wt % to about 60.0 wt %. The weight percent of the content of the solvents and/or carrier fluids in the reaction mixtures can based on the total weight of the reaction mixtures.

The one or more reaction mixtures can be heated to a temperature from a low of about 0 °C, about 15 °C, and about 25 °C, to a high of about 35 °C, about 65 °C, and about 200 °C For example, the one or more reaction mixtures can be heated to a temperature from about 25 °C to about 28 °C, about 25 °C to about 35 °C, about 25 °C to about 90 °C, about 30 °C to about 45 °C, about 450 °C to about 55 °C, about 40 °C to about 90 °C, about 43 °C to about 78 °C, about 40 °C to about 90 °C, about 100 °C to about 200 °C. In another example, the one or more reaction mixtures can be at room temperature. In another example, the reaction occurs at a temperature of greater than about 40 °C or greater than about 50 °C. The first reaction mixture, second reaction mixture, third reaction mixture, and higher iterations and reaction mixtures can be performed at different temperatures.

The first reaction mixture, second reaction mixture, third reaction mixture, and more reaction mixtures can be reacted and/or stirred for a first reaction time, second reaction time, third reaction time, and higher iterations of reaction times from a short of about 15 s, about 120 s, or about 300 s, to a long of about 1 h, about 24 h, or about 72 h. For example, the first reaction mixture, second reaction mixture, third reaction mixture, and higher iterations of reaction mixtures can be reacted and/or stirred for a first reaction time, second reaction time, third reaction time, and higher iterations of reaction times can be from about 1 min to about 15 min, about 5 min to about 45 min, about 1 h to about 12 h, about 5 h to about 15 h, about 8 h to about 10 h, about 14 h to about 16 h, about 15 h to about 16 h, about 10 hours to about 24 hours, about 12 h to about 17 h, about 12 h to about 24 h, about 22 h to about 50 h, or about 24 h to about 72 h.

The one or more reaction mixtures can be reacted and/or stirred in an open reaction container or a closed container. The one or more reaction mixtures can be reacted and/or stirred under a vacuum. The one or more reaction mixtures can be reacted and/or stirred under an inert atmosphere, such as He, Ne, N₂, and Ar.

The one or more reactions mixtures can have a viscosity that varies widely. For example, the one or more reactions mixtures can have a viscosity from a low of about 1.0 cP, about 10.0 cP, or about 1,000.0 cP, to a high of about 25,000.0 cP, about 90,000.0 cP, or about 250,000.0 cP. In another example, the reactions mixtures can have a viscosity from about 1.0 cP to about 250,000.0 cP, about 2.0 cP to about 100.0 cP, about 25.0 cP to about 2,500.0 cP, about 2,500.0 cP to about 200,000.0 cP, about 10,000.0 cP to about 100,000.0 cP, about 10,000.0 cP to about 50,000.0 cP, about 100,000.0 cP to about 250,000.0 cP, about 6,000.0 cP to about 85,000.0 cP, about 7,000.0 cP to about 75,000.0 cP, about 7,000.0 cP to about 80,000.0 cP, about 5,000.0 cP to about 10,000.0 cP, or about 50,000.0 cP to about 200,000 cP. The viscosity of the reaction mixtures can be measured on a Brookfield viscosimeter. The viscosity of the reaction mixtures can be measured at various temperatures, such as 25 °C, 40 °C, 60 °C, and 100 °C.

The one or more solvent mixtures can have a viscosity that varies widely. For example, the one or more solvent mixtures can have a viscosity from a low of about 1.0 cP, about 10.0 cP, or about 1,000.0 cP, to a high of about 25,000.0 cP, about 90,000.0 cP, or about 250,000.0 cP. In another example, the solvent mixtures can have a viscosity from about 1.0 cP to about 250,000.0 cP, about 2.0 cP to about 100.0 cP, about 25.0 cP to about 2,500.0 cP, about 2,500.0 cP to about 200,000.0 cP, about 10,000.0 cP to about 100,000.0 cP, about 10,000.0 cP to about 50,000.0 cP, about 100,000.0 cP to about 250,000.0 cP, about 6,000.0 cP to about 85,000.0 cP, about 7,000.0 cP to about 75,000.0 cP, about 7,000.0 cP to about 80,000.0 cP, about 5,000.0 cP to about 10,000.0 cP, or about 50,000.0 cP to about 200,000 cP. The viscosity of the solvent mixtures can be measured on a Brookfield viscosimeter. The viscosity of the reaction mixtures can be measured at various temperatures, such as 25 °C, 40 °C, 60 °C, and 100 °C.

The one or more compositions can have a viscosity that varies widely. For example, the one or more compositions can have a viscosity from a low of about 1.0 cP, about 10.0 cP, or about 1,000.0 cP, to a high of about 25,000.0 cP, about 90,000.0 cP, or about 250,000.0 cP. In another example, the compositions can have a viscosity from about 1.0 cP to about 250,000.0 cP, about 2.0 cP to about 100.0 cP, about 25.0 cP to about 2,500.0 cP, about 2,500.0 cP to about 200,000.0 cP, about 10,000.0 cP to about 100,000.0 cP, about 10,000.0 cP to about 50,000.0 cP, about 100,000.0 cP to about 250,000.0 cP, about 6,000.0 cP to about 85,000.0 cP, about 7,000.0 cP to about 75,000.0 cP, about 7,000.0 cP to about 80,000.0 cP, about 5,000.0 cP to about 10,000.0 cP, or about 50,000.0 cP to about 200,000 cP. The viscosity of the compositions can be measured on a Brookfield viscosimeter. The viscosity of the compositions can be measured at various temperatures, such as 25 °C, 40 °C, 60 °C, and 100 °C.

The pharmaceutically acceptable carriers or diluents for use with compounds of Formula I can include, but are not limited to: water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these.

The adjuvants can include, but are not limited to, wetting agents, stabilizing agents, binding agents, dispersing agents, emulsifying agents, suspending agents, bioadhesive agents, polymers, and flavoring agents. The preservative can include, but is not limited to benzalkonium chloride. The polymer can include, but is not limited to, polyacrylic acid, polyvinyl alcohol, hyaluronic acid, hydroxypropylmethyl cellulose, hydroxyethyl cellulose, methyl cellulose, and heteropolysaccharide polymers, such as gelan gum.

To provide for the administration of such dosages for the desired therapeutic treatment, pharmaceutical compositions of the compounds of Formula I can be from a low of about 0.05% and to a high of about 99.9% by weight of the total of one or more of the compounds or pharmaceutically acceptable salts of Formula I based on the weight of the total composition, including a pharmaceutically acceptable carrier and any adjuvant. For example, the pharmaceutical compositions of compounds or pharmaceutically acceptable salts of Formula I can have compounds or pharmaceutically acceptable salts of Formula I from about 0.05 wt % to about 99.9 wt %, 0.05 wt % to about 10 wt %, 0.2 wt % to about 5 wt %, 1 wt % to about 20 wt %, 5 wt % to about 45 wt %, 8 wt % to about 65 wt %, or 15 wt % to about 85 wt %, based on the weight of the total composition, including any pharmaceutically acceptable carrier and/or adjuvant.

The pharmaceutically acceptable carriers can be a solid, a liquid, or both, and may be formulated with the compound as a unit-dose composition, for example, a tablet, which can contain from a low of about 0.05 wt % to a high of about 99.9 wt % f the active compounds. The pharmaceutical compositions of compounds of Formula I can be coupled with suitable polymers as targetable pharmaceutically acceptable carriers.

Other pharmaceutically acceptable carriers and modes of administration known in the pharmaceutical art may also be used. Pharmaceutical compositions of compounds of Formula I may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

The compounds of Formula I can be used alone or in combination with other therapeutic and/or active compounds in the treatment of various conditions or disease states. The compounds of Formula I and other compounds may be administered simultaneously (either in the same dosage form or in separate dosage forms) or sequentially. The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration. The phrases “concurrent administration,” “co-administration,” “simultaneous administration,” and “administered simultaneously” mean that the compounds are administered in combination.

The compounds of Formula I may be administered by any suitable route, preferably in a form of a pharmaceutical composition adapted to such a route, and in a dose effective for the treatment intended. The active compounds and compositions, for example, may be administered orally, rectally, parenterally, or topically (e.g., intranasal or ophthalmic).

The compounds of Formula I and compositions thereof can be administered to a subject orally, topically, and/or by injection. Oral administration may involve swallowing, so that the compound enters the gastrointestinal tract, or buccal or sublingual administration may be employed, by which the compound enters the blood stream directly from the mouth.

The compounds of Formula I may be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

The compounds of Formula I can be formulated such that administration topically to the skin or mucosa (i.e., dermally or transdermally) leads to systemic absorption of the compounds. The compounds of Formula I can also be formulated such that administration intranasally or by inhalation leads to systemic absorption of the compound.

The dosage regimen for the compounds of Formula I can be based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions. The total daily dose of a compounds of Formula I (administered in single or divided doses) is typically from about 0.01 mg/kg to about 100 mg/kg. In another embodiment, the total daily dose of the compound of Formula I is from about 0.1 mg/kg to about 50 mg/kg, and in another embodiment, from about 0.5 mg/kg to about 30 mg/kg (i.e., mg compound of Formula I per kg body weight). In one embodiment, dosing is from about 0.01 mg/kg/day to about 10 mg/kg/day. In another embodiment, dosing is from about 0.1 mg/kg/day to about 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day may be used to increase the total daily dose, if desired. Intravenously, doses may range from about 0.1 to about 10 mg/kg/minute during a constant rate infusion.

Oral administration of a solid dose form can be presented in discrete units, such as hard or soft capsules, pills, cachets, lozenges, or tablets, each containing a predetermined amount of at least one compound of Formula I. In another embodiment, the oral administration may be in a powder or granule form. In another embodiment, the oral dose form is sub-lingual, such as, for example, a lozenge. In such solid dosage forms, the compounds of Formula I are ordinarily combined with one or more adjuvants. Such capsules or tablets may contain a controlled-release formulation. In the case of capsules, tablets, and pills, the dosage forms also may comprise buffering agents or may be prepared with enteric coatings.

For oral administration, the compounds of Formula I can be provided in the form of tablets containing from about 0.01 mg to about 0.05 mg, about 0.1 mg to about 0.5 mg, about 1.0 mg to about 2.5 mg, about 5.0 mg to about 10.0 mg, about 15.0 mg to about 25.0 mg, about 50.0 mg to about 75.0 mg, about 100 mg to about 125 mg, 150 mg to about 175 mg, about 200 mg to about 250 mg, and 0.01 mg to about 500 mg of the active ingredient for the symptomatic adjustment of the dosage to the patient. In another example, the compounds of Formula I can be provided in the form of tablets containing from about 0.05 wt % to about 99.9 wt %, 0.05 wt % to about 10 wt %, 0.2 wt % to about 5 wt %, 1 wt % to about 20 wt %, 5 wt % to about 45 wt %, 8 wt % to about 65 wt %, or 15 wt % to about 85 wt % f compounds of Formula I, based on the weight of the total composition, including any pharmaceutically acceptable carrier and/or adjuvant.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). For example, the compounds of Formula I can be provided in a liquid form containing from about 0.05 wt % to about 99.9 wt %, 0.05 wt % to about 10 wt %, 0.2 wt % to about 5 wt %, 1 wt % to about 20 wt %, 5 wt % to about 45 wt %, 8 wt % to about 65 wt %, or 15 wt % to about 85 wt % f compounds of Formula I, based on the weight of the total composition, including any pharmaceutically acceptable carrier and/or adjuvant.

In another embodiment, the pharmaceutical compositions of the compounds of Formula I can include a parenteral dose form. Parenteral administration can include, but is not limited to, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using suitable dispersing, wetting, and/or suspending agents, and include depot formulations.

The compounds of Formula I can be administered a topical dose form. Topical administration can include, but is not limited to, transdermal administration, such as via transdermal patches or iontophoresis devices, intraocular administration, or intranasal or inhalation administration. Compositions for topical administration can include, but is not limited to, topical gels, sprays, ointments, and creams. A topical formulation can include a compound that enhances absorption or penetration of the active ingredient through the skin or other affected areas. When the compounds of Formula I are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes can also be used. Typical carriers include alcohol, water, mineral oil, liquid petrolatum, white petrolatum, glycerin, polyethylene glycol and propylene glycol. Penetration enhancers may be incorporated-see, for example, Finnin and Morgan, J. Pharm. Sci., 88 (10), 955-958 (1999).

For intranasal administration or administration by inhalation, the active compounds of Formula I are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone; as a mixture, for example, in a dry blend with lactose; or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may include a bioadhesive agent, for example, chitosan or cyclodextrin.

The compounds of Formula I can be used in kits that are suitable for use in performing the methods of treatment described above. In one embodiment, the kit contains a first dosage form comprising one or more of the compounds of the present invention and a container for the dosage, in quantities sufficient to carry out the methods of the present invention.

The compounds of Formula I can also be administered utilizing liposome technology, slow release capsules, implantable pumps, and biodegradable containers. These delivery methods can, advantageously, provide a uniform dosage over an extended period of time.

Various techniques may be used to increase bioavailability of the compounds of Formula I. For example, prodrugs of compounds of Formula I can be prepared. Prodrugs employ various physical and chemical modifications to improve features of the active drug, and in some embodiments may be viewed as pharmacologically inactive prodrug functional groups that undergo a chemical transformation or enzymatic cleavage to liberate the active parent drug and produce the desired effect in the body. Utilizing a prodrug approach can yield benefits such as enhanced solubility, improved selective targeting of drugs to anatomical sites, protection from rapid metabolism and elimination, reduction toxic effects of an active drug on other parts of the body, and enhanced patient compliance.

Non-limiting examples of techniques useful for enhancing the bioavailability of compounds or pharmaceutically acceptable salts of Formula I can include the use of co-solvents, hydrotropy, micronization, change in dielectric constant of solvent, amorphous forms, chemical modification of the drug, use of surfactants, inclusion complex, alteration of pH of solvent, use of hydrates or solvates, use of soluble prodrugs, application of ultrasonic waves, functional polymer technology, controlled precipitation technology, evaporative precipitation in aqueous solution, use of precipitation inhibitors, solvent deposition, precipitation, selective adsorption on insoluble carriers, size reduction technologies, lipid based delivery systems, micellar technologies, porous micro particle technology, solid dispersion technique, and various types of solid dispersion systems.

Other methods for enhancement of bioavailability, such as by the enhancement of solubility, are described in Reddy M. S. et al., “Solubility enhancement of fenofibrate, a BCS class II drug, by self-emulsifying drug delivery systems,” International Research Journal of Pharmacy, 2011, 2(11): 173-177; Khamkar G S, “Self micro emulsifying drug delivery system (SMEED) o/w microemulsion for BCS Class II drugs: an approach to enhance oral bioavailability,” International Journal of Pharmacy and Pharmaceutical Sciences, 2011,3(3):1-3; Elgart A et al., “Improved oral bioavailability of BCS class 2 compounds by self nano-emulsifying drug delivery systems (SNEDDS): the underlying mechanisms for amiodarone and talinolol”, Pharm Res., 2013 December; 30(12):3029-44; Singh N. et al., “Techniques for bioavailability enhancement of BCS class II drugs: a review,” International Journal of Pharmaceutical and Chemical Science, 2013, 2(2):1092-1101; Elkihel L. et al., “Synthesis and orally macrofilaricidal evaluation of niclosamide lymphotropic prodrugs,” Arzneimittelforschung, 1994, 44(11): 1259-64; and Kansara H. et al., “Techniques used to enhance bioavailability of BCS class II drugs: a review,” Int. J. Drug Dev. & Res., 2015, 7(1):82-93.

The method of using compounds or pharmaceutically acceptable salts of Formula I can include, but is not limited to, packaged dosage formulations and kits. A packaged dosage formulation can including one or more containers of one or more compounds of Formula I formulated in a pharmaceutically acceptable dosage. The package can contain discrete quantities of the dosage formulation, such as tablet, capsules, lozenge, and powders. The quantity of compound in a dosage formulation and that can be administered to a patient can vary from about 1 mg to about 5000 mg, about 1 mg to about 2000 mg, about 1 mg to about 500 mg, about 5 mg to about 250 mg, or about 10 mg to about 100 mg.

The kits can be packaged into suitable packaging material, optionally in combination with instructions for using the kit components, e.g., instructions for using compounds of Formula I. In one embodiment, a kit includes an amount of at least one compound of Formula I, and instructions for administering at least one compound of Formula I to a subject in need of treatment on a label or packaging insert. In further embodiments, a kit includes an article of manufacture, for delivering at least one compound of Formula I into a subject locally, regionally or systemically.

As used herein, the term “packaging material” refers to a physical structure housing the components of the kit. The packaging material can maintain the components in a sterile state, and can be made of material commonly used for such purposes (e.g., paper, corrugated fiber, glass, plastic, foil, ampules, etc.). The label or packaging insert can include appropriate printed and/or digital instructions, for example, for practicing a method of the invention. Thus, in additional embodiments, a kit includes a label or packaging insert including instructions for practicing a method of the invention in solution, in vitro, in vivo, or ex vivo.

Instructions can therefore include instructions for practicing any of the methods of using compounds of Formula I described herein. For example, pharmaceutical compositions can be included in a container, pack, or dispenser together with instructions for administration to a subject to treat a disease. Instructions may additionally include indications of a satisfactory clinical endpoint or any adverse symptoms that may occur, storage information, expiration date, or any information required by regulatory agencies such as the Food and Drug Administration or European Medicines Agency for use in a human subject.

The instructions may be digital or on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (diskette or hard disk), optical CD such as CD- or DVD-ROM/RAM, magnetic tape, electrical storage media such as RAM and ROM and hybrids of these such as magnetic/optical storage media.

Each component of the kit can be enclosed within an individual container or in a mixture, and all of the various containers can be within single or multiple packages. Kits can include packaging material that is compartmentalized to receive one or more containers such as vials, tubes, and the like, each of the container(s) including one of the separate elements to be used in a method described herein. Packaging materials for use in packaging pharmaceutical products include, by way of example only U.S. Pat. Nos. 5,323,907, 5,052,558 and 5,033,252. Examples of pharmaceutical packaging materials can include, but are not limited to, blister packs, bottles, tubes, pumps, bags, vials, light-tight sealed containers, syringes, bottles, and any packaging material suitable for a selected formulation and intended mode of administration and treatment.

A kit may include one or more additional containers, each with one or more of various materials desirable from a commercial and user standpoint for use of the compounds for treating or preventing diseases. Non-limiting examples of such materials include, but not limited to, buffers, diluents, carrier, package, container, vial and/or tube labels listing contents and/or instructions for use, and package inserts with instructions for use.

A label can be on or associated with a container containing a compounds of Formula I. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In some embodiments of the kit, the compounds of the Formula I can be presented in a pack or dispenser device which can contain one or more unit dosage forms containing a compound disclosed herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutically acceptable carrier can also be prepared, placed in an appropriate container, and labeled for treatment of an indicated condition.

### EXAMPLES

In order to provide a better understanding of the foregoing discussion, the following non-limiting examples are offered. Although the examples can be directed to specific embodiments, they are not to be viewed as limiting the invention in any specific respect.

### Cells, Reagents, and Antibodies

Human embryonic kidney (HEK293) and Calu-3 cells were purchased from ATCC Vero E6-TMPRSS2-T2A-ACE2 was obtained through BEI Resources, NIAID. The cells were maintained in Dulbecco's Modified Eagle Medium (DMEM) supplemented with 10% fetal bovine serum (FBS, Sigma-Aldrich), 2 mM L-glutamine, and antibiotics (25 U/mL penicillin and 25 μg/mL streptomycin). For Vero E6-TMPRSS2-T2A-ACE2 cells, the medium also included 10 μg/mL puromycin (InvivoGen). The cells were incubated at 37 °C in a humidified environment with 5% CO₂. The following antibodies were used in this study: SARS-CoV-2 NSP12 polyclonal antibody, SARS-CoV-2 NSP8 polyclonal antibody, SARS-CoV-2 NSP7 polyclonal antibody, GAPDH monoclonal antibody as a loading control, HCoV-OC43 nucleoprotein and spike antibody and HCoV-229E nucleoprotein and spike antibody. (Provide the cat. # and the company name of these antibodies).

### Viruses

The SARS-CoV-2 USA-WA1/2020, HCoV-OC43, and HCoV-229E strains were obtained through BEI Resources, NIAID/NIH. In-house virus stock was prepared using ATCC/BEI virus stocks by inoculating a monolayer of Calu-3 human lung adenocarcinoma cells (ATCC, cat. #HTB-55). These cells were cultured in DMEM (Corning, cat. #10-013-CM) supplemented with 10% fetal bovine serum (FBS, Sigma-Aldrich, cat. #F2442) and Penicillin/Streptomycin (Fisher Scientific, cat. #SV30010). Four days post-infection, the Calu-3 monolayer exhibited characteristic cytopathic effects consistent with SARS-CoV-2 infection. The culture supernatant was collected, centrifuged to remove cell debris, and designated as P1 stock. The virus stock was aliquoted and stored at −80 °C. All SARS-CoV-2 propagation and infection assays were conducted in the state-of-the-art Biological Safety Level 3 (BSL-3) containment laboratory at the University of Nevada, Reno. These experiments adhered to rigorously approved protocols and procedures established by the Institutional Biosafety Committee (IBC) of UNR, ensuring the highest safety and compliance standards. HCoV-OC43 was cultured in Vero E6-TMPRSS2-T2A-ACE2 cells and HCoV-229E in MRC-5 cells. The incubation and collection procedures followed the steps described above for SARS-CoV-2, following approved protocols and procedures established by the Institutional Biosafety Committee (IBC) of UNR for the Biological Safety Level 2 agents.

### Cell-Based SARS-CoV RdRp Activity Assay

HEK293 cells were seeded in 6-well plates for 3 hours until properly attached and then transiently transfected with the RdRP-reporter plasmid, pLVX-EF1α-IRES-Puro, containing the SARS-related coronavirus 2 (USA-WA1/2020) non-structural protein 12, 8, and 7 genes (BEI Resources, NIAID, NIH) using the 293 Fectin™ reagent (Gibco by Thermo Scientific) following the manufacturer's instructions. After 48 hours, the cells were trypsinized, counted, and reseeded in a 24-well plate to treat with test compounds or DMSO (as a control). The cells were incubated at 37 °C for 48 hours with the compounds. Firefly (FLuc) and Renilla (RLuc) luciferase activities were measured using the Dual-Luciferase® Reporter Assay System (Promega Corporation) according to the manufacturer's instructions. The relative activity of RdRP was determined by normalizing the FLuc level to that of RLuc (FLuc/RLuc).

### Western Blotting

Sample preparation for Western blotting, cell transfection, and treatment procedures were the same as above. For HCoV-OC43 and HCoV-229E, Vero E6-TMPRSS2-T2A-ACE2 cells (150,000/well) were seeded in 24-well plates. After cell attachment (3 h), the cells were infected with the respective viruses and incubated for 2 h. Then, the cells were incubated with the test compounds (10 μM), including DMSO as a control, and incubated for 48 h. After incubation, the cells were harvested for protein extraction. The treated cells were washed with chilled PBS, then lysed in RIPA lysis buffer supplemented with protease inhibitors (phenylmethylsulfonyl fluoride (1 mM), aprotinin (1 μg/mL), pepstatin (1 μg/mL), sodium fluoride (1 μg/mL), and leupeptin (1 μg/mL)). The harvested lysates were incubated on ice for 30 minutes. The cell lysates were sonicated using a probe sonicator; then, the cell debris was removed by centrifugation (12,000 rpm for 10 minutes at 4 °C). The clear lysates were transferred to centrifuge tubes for the western blot experiment. Protein samples were resuspended in SDS-PAGE loading buffer, denatured at 95 °C for 5 minutes, and resolved by SDS-PAGE. The proteins of interest were then transferred to a 0.45 μM nitrocellulose membrane using standard protocols (Bio-Rad Laboratories, Hercules, CA, United States). Proteins were detected by incubating the membrane with specific primary antibodies, followed by incubation with secondary antibodies, and scanned using the ChemiDoc MP Imaging System (Bio-Rad).

### Dose-Response Assays

The transiently transfected HEK293 cells transfected with the reporter (R) and NSP7-NSP8-NSP12 plasmids were seeded at 150,000 cells per well in 24-well plates (transfection method described above) and allowed to attach for 3 hours. The cells were then treated with different concentrations of the compound TK018 (100 μM, 50 μM, 25 μM, 12.5 μM, 6.25 μM, 3.125 μM, and 1.5625 μM). After 48 hours of incubation at 37 °C with 5% CO₂, the compound was removed by washing the cells with 1×PBS. The treated cells were lysed in 100 μL of passive lysis buffer, and a dual-luciferase reporter assay was performed. The absolute half-maximal inhibitory concentration (IC50) was calculated using the Quest Graph™ IC50 calculator (v.1) [https://www.aatbio.com/tools/ic50-calculator-v1].

For the SARS-CoV-2 and HCoV-OC43 infection assays, Vero E6-TMPRSS2-T2A-ACE2 cells were seeded at 150,000 cells per well in 24-well plates and allowed to attach for 3 hours. The cells were infected with USA-WA1/2020 and HCoV-OC43 for 2 hours, followed by treatment with the same concentrations of TK018 as described above. After 48 hours of treatment, RNA was extracted from the cells, and reverse transcription-quantitative PCR (RT-qPCR) was performed to quantify the amount of viral genome copies. The absolute half-maximal inhibitory concentrations (IC50) of TK018 for USA-WA1/2020 and HCoV-OC43 were calculated based on the remaining viral load in the cells at each concentration using the Quest Graph™ IC50 calculator (v.1). All experiments were conducted in triplicate. The resulting data, including curves and graphs, were generated using GraphPad Prism™.

### Cell Viability (MIT) Assay

Cell viability following compound treatment was determined using the standard colorimetric MTT assay [3-(4,5-dimethylthiazol-2-yl)-2,5-diphenyltetrazolium bromide](Thermo Fisher Scientific), according to the manufacturer's instructions. HEK293 cells transfected with the reporter (R) and NSP7-NSP8-NSP12 plasmids and Vero-TMPRSS2 cells were seeded at 30,000 cells per well in a 96-well microplate. After 3 hours of incubation at 37 °C with 5% CO2, the cells were treated with varying concentrations of the compound TK018 (100 μM, 50 μM, 25 μM, 12.5 μM, 6.25 μM, 3.125 μM, and 1.5625 μM) for 48 hours. The cells were then washed with 1×PBS, and 100 μL of 0.5 mg/mL MTT solution was added to each well, followed by incubation at 37 °C for 4 hours. The culture medium (50 μL) was used as a negative control (blank). After incubation, once intracellular purple formazan crystals were visible under the microscope; the MTT solution was removed and replaced with 50 μL of solubilizing DMSO solution. The cells were incubated at room temperature for 30 minutes to dissolve the formazan crystals. The absorbance was measured using a GloMax® Explorer microplate reader (Promega Corporation) at a wavelength of 570 nm. The background absorbance from wells containing only medium (Absblank) was subtracted from all wells, and the percentage of viable cells was calculated using the following equation: % viable cells=[(Absₛₐₘₚₗₑ−Abs_{blank})/(Abs_{control}−Abs_{blank})]*100.

### Quantification of the Viral Genome Copies in the Infected Cells Treated With Compounds

Vero E6-TMPRSS2-T2A-ACE2 cells (150,000 cells per well) were seeded in 24-well plates and incubated at 37 °C with 5% CO₂ for 3 hours to allow cell attachment. After attachment, the monolayer cells were infected with SARS-CoV-2, HCoV-OC43, or HCoV-229E for 2 hours. Following infection, unattached viruses were removed by washing the cells with PBS, and the media was replaced with either DMSO (control) or the test compounds (10 μM) containing medium. The infected and treated cells were incubated for 48 hours at 37 °C with 5% CO₂. Following treatment, the impact on viral replication was evaluated by quantifying viral genome copies using RT-qPCR. This analysis measured intracellular genome copies and cell-free virions in the culture supernatant. Additionally, the supernatant from treated and control cells was used to perform a plaque assay, directly assessing cell-free infectious virions. These combined approaches comprehensively evaluated the treatment's effect on viral replication.

### RNA Extraction and RT-qPCR

For detection of viral genomic RNA post-infection and compound treatment via rt-qPCR. Total RNA was extracted from supernatants (100 μL) and cells treated from control (DMSO) or compounds, using Trizol reagent (Invitrogen, Carlsbad, CA, USA) via Direct-zol RNA extraction kit (Zymo Research), according to the manufacturer's protocol. An aliquot of the extracted total RNA (5 μL) was used for the relative quantification of viral genomic copies. RT-qPCR was done using TaqPath and specific primer-probe for SARS-CoV-2, HCoV-OC43, and HCoV-229E via QuantStudio 5 rt-qPCR system (Thermo Fisher Scientific, Waltham, MA, USA). You should mention the cat # of the Taqman Assay.

### Plaque Assay

Plaque assay was performed as described previously. Briefly, the Vero E6-TMPRSS2-T2A-ACE2 cells (150,000 cells per well) were seeded in 24-well plates and allowed to attach for 3 hours. After attachment, the monolayers were infected with the indicated volumes of the supernatant from the compounds of DMSO-treated cells for 2 hours. Following the incubation, the medium was replaced with 2 mL of a 1.5% CMC/DMEM overlay medium, and the cells were further incubated at 37 °C in 5% CO2. The incubation times were 3 days for the SARS-CoV-2 and 5 days for HCoV-OC43 and HCoV-229E. After the incubation, the overlay medium was removed and plates were washed with 1×DPBS, followed by adding 1.0 ml of 10% formaldehyde (Sigma-Aldrich, cat. #F8775) in 1×DPBS and incubating for 30 min at room temperature. Next, the cells were stained with 0.2% crystal violet solution in 20% ethanol (500 μL) at room temperature for 15 min. After removing the staining solution, each well was washed thrice with sterile distilled water. The plates were then inverted and left to dry on an absorbent pad for 2 h. Plaques, appearing as clear circles in the purple monolayer, were counted using a negative control with no plaques as a reference. The plaque-forming units (PFU per mL) were calculated based on the number of plaques for each dilution.

PFU/mL=average number of plaques/dilution factor of the well×volume of inoculum per plate]

Cell-based RdRp assay for screening compounds to identify the inhibitors of SARS-CoV-2 RdRp activity were used (31). The RdRp-luciferase reporter plasmid was co-transfected with RTC (Replication-Transcription Complex) proteins expressing plasmids (nsp7, nsp8, and nsp12) into HEK293 cells using the 293 Fectin™ reagent. Cells harboring the dual-reporter construct were seeded into 24-well microplates and treated with compounds at a final concentration of 10 μM or DMSO as a vehicle control. The system utilizes two luciferase reporters, firefly luciferase (FLuc) and renilla luciferase (RLuc), for the RdRp activity and normalization, respectively. The FLuc reporter is regulated by the 5′-untranslated region (5′-UTR) of the SARS-CoV-2 genome and is modulated by RdRp activity. This design allows the FLuc signal to reflect the activity of RdRp, with decreased FLuc expression indicating effective inhibition of RdRp by the test compounds. The RLuc reporter, independent of RdRp, serves as a stable internal control to normalize variations in cell number, viability, or other non-specific effects of treatment. At 48 hours post-treatment, FLuc, and RLuc activities were measured using a dual-luciferase assay system. The ratio of FLuc activity to RLuc activity (FLuc/RLuc index) was calculated to quantify the dR activity. A reduction in the FLuc/RLuc index corresponded to a proportional decrease in RdRp function, indirectly measuring the compound's inhibitory potency.

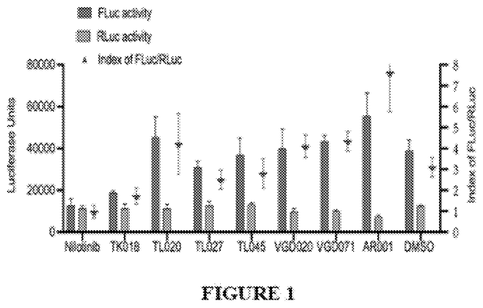

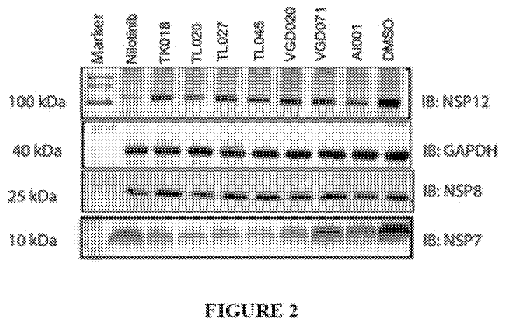

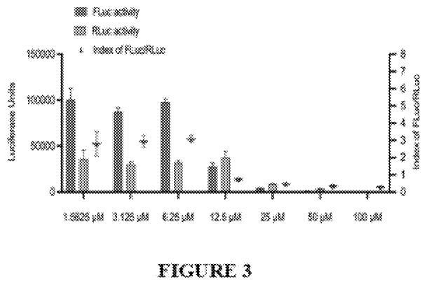

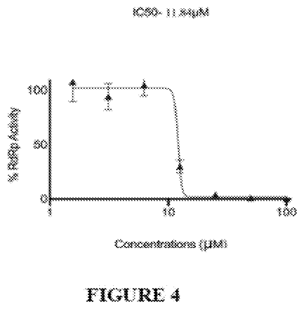

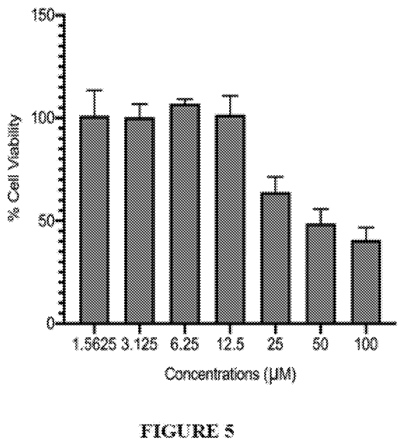

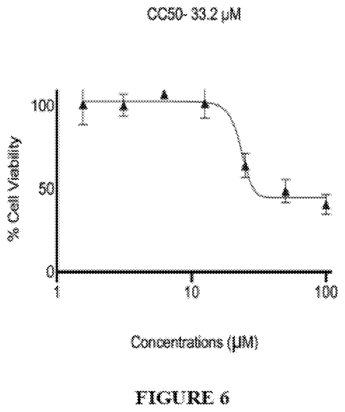

As illustrated in FIG. 1, among the tested compounds, Nilotinib and TK018 demonstrated significant inhibition of SARS-CoV-2 RdRp activity, reducing the FLuc/RLuc index by more than 50%. To further characterize the effects of these compounds, the expression levels of RTC proteins, nsp12, nsp8, and nsp7, alongside the loading control GAPDH in transiently transfected and treated cells (FIG. 2) were evaluated. Cells were treated with the respective compounds, including DMSO as a control. Cells treated with Nilotinib showed reduced expressions of all the RTC proteins, compared to the DMSO-treated cells (FIG. 2). This finding aligns with the results of a cell toxicity assay, which revealed that Nilotinib exhibits substantial toxicity in transiently transfected cells. IC50 analysis further confirmed that Nilotinib's effects are associated with non-specific toxicity, leading to broad suppression of both FLuc and RLuc levels. While these data were not shown here, the observed toxicity renders Nilotinib unsuitable as a specific RdRp inhibitor. Notably, other tested compounds, including TK018, effectively reduced the expression of nsp12, nsp8, and nsp7 (FIG. 2). Through the combined analysis of reporter assay and western blot data, TK018 emerged as a potent inhibitor of RdRp activity, also demonstrating a reduction in RdRp protein expression (FIG. 1). Further investigation into the antiviral activity of TK018 was performed using a dose-dependent analysis on transiently transfected cells. The half-maximal inhibitory concentration IC50 for RdRp inhibition was determined to be 11.84 μM (FIGS. 3-4), demonstrating robust efficacy. Additionally, cytotoxicity evaluation of TK018 in HEK293L cells using an MTT assay revealed low cytotoxicity, with a CC50 of 33.2 μM (FIGS. 5-6). This colorimetric assay provides a sensitive and accurate method for determining cell viability by generating an insoluble formazan (purple color) from the water-soluble tetrazolium MTT dye. Among all tested compounds, TK018 stood out as a potent and specific SARS-CoV-2 RdRp inhibitor with promising antiviral activity, while Nilotinib was excluded due to its significant cytotoxic effects. These findings position TK018 as a promising therapeutic candidate for further development.

TK018 Suppressed the Replication of SARS-CoV-2 in an In-Vitro Infection Assay

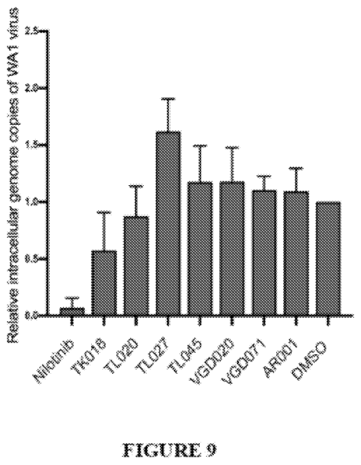

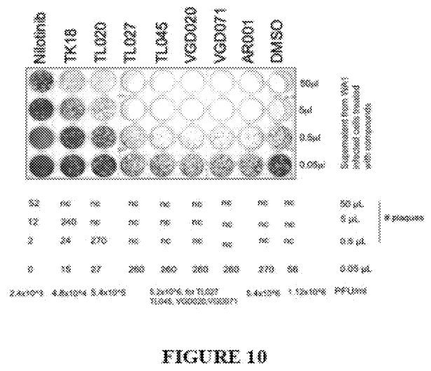

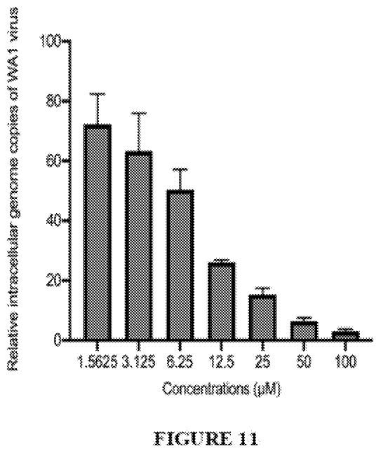

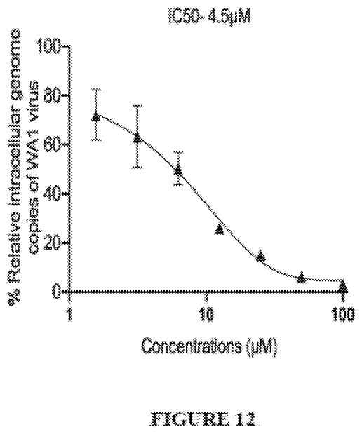

The antiviral activity of nilotinib on SARS-CoV-2 replication in-vitro using the USA-WA1/2020 strain of SARS-CoV-2 propagated in Calu-3 lung epithelial cells was further evaluated. The antiviral effects of Nilotinib and other CADA compounds were assessed by infecting Vero-TMPRSS2 cells with the SARS-CoV-2 USA-WA1/2020 strain. After a 2-hour incubation (at 37 °C and 5% CO2), cells were treated with 10 μM concentrations of Nilotinib, various CADA compounds, or DMSO as a control. Following 48 hours of treatment, intracellular viral RNA was extracted, and SARS-CoV-2 genomic RNA copies were quantified using RT-qPCR (FIG. 9). Additionally, infectious viral particles in the supernatants were quantified in a plaque assay by infecting the Vero-TMPRSS2 cell monolayers. Varying amounts of supernatants (50 μL, 5 μL, 0.5 μL, and 0.05 μL) were used for determining the plaque-forming units. After 3 days of CMC-MEM overlay, the cell monolayers were fixed and stained with crystal violet to visualize and count the plaque-forming units (PFU/mL). TK018 and TL020 significantly inhibited SARS-CoV-2 replication and propagation compared to the DMSO control, as evidenced by the reduced viral genomic copies and number of plaques (FIGS. 9-10). However, cells treated with Nilotinib showed complete suppression of viral genomic copies and plaque formation, not due to antiviral efficacy but because of cytotoxicity, as indicated by the absence of plaques (FIGS. 9-10). To further quantify the antiviral activity of TK018, its half-maximal inhibitory concentration (IC50) was determined. Vero-TMPRSS2 cells infected with the USA-WA1/2020 strain were treated with varying concentrations of TK018, and viral genomic RNA copies were measured after 48 hours. TK018 effectively reduced SARS-CoV-2 genome levels in a dose-dependent manner, with an IC50 value of 4.5 μM (FIGS. 11-12), demonstrating its potent antiviral activity. MTT assay assessed whether TK018 exhibited cytotoxic effects on Vero-TMPRSS2 cells. This colorimetric assay measuring the cell viability by converting water-soluble MTT dye into insoluble purple formazan, revealed no significant cytotoxicity up to a concentration of 56.85 M, even after 48 hours of treatment. These findings suggested that TK018 possesses antiviral activity without any cytotoxicity. Overall, TK018 demonstrated robust antiviral activity against SARS-CoV-2, significantly reducing viral replication in an in vitro cell culture model without causing cytotoxicity, making it a promising candidate for further development.

### CADA Compounds Suppressed the Replication of Beta Human Coronaviruses, HCoV-OC43, In In-Vitro-Infected Cells

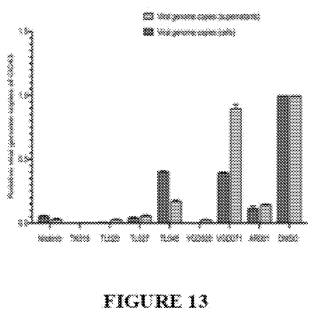

Next, the antiviral efficacy of these CADA compounds against another human coronavirus, HCoV-OC43, by conducting in vitro infection of Vero-TMPRSS2 cells, was evaluated. HCoV-OC43 virus prepared in-house was used to infect Vero-TMPRSS2 cells for 2 hours at 37 °C with 5% CO₂. Following infection, the cells were treated with 10 μM concentrations of various CADA compounds, including Nilotinib, TK018, TL020, TL027, VGD020, AR001, and DMSO as a control. After 48 hours of treatment, viral RNA was extracted from both cells and supernatants, and the levels of HCoV-OC43 RNA genomes were quantified using an RT-qPCR assay (FIG. 13). A significant reduction in the number of viral genome copies in both the supernatant and the cells among compound-treated cells demonstrating antiviral activity was detected. Notably, TK018, TL020, TL027, and VGD020 exhibited potent inhibition and reduction in the viral genome copies.

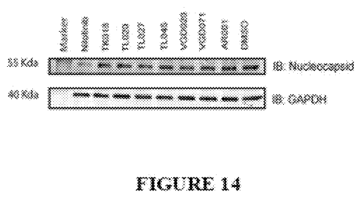

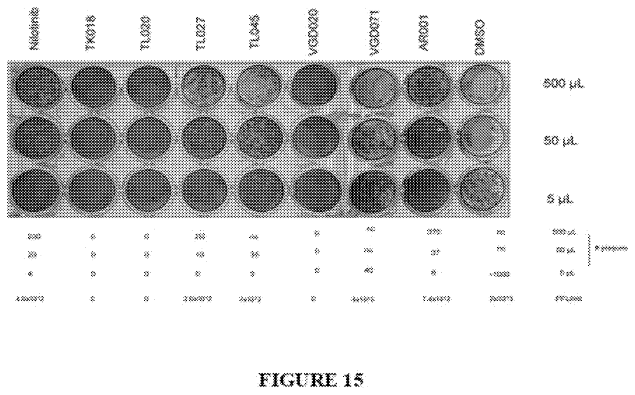

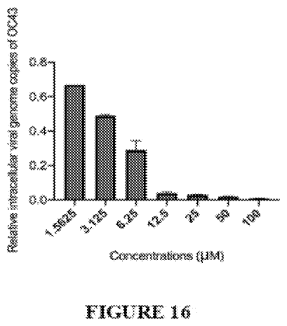

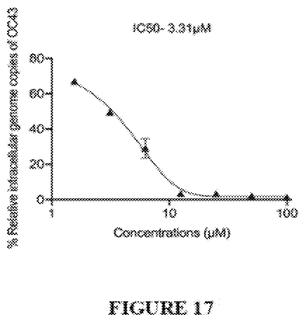

To corroborate these findings, western blot analysis was performed to measure the expression of the HCoV-OC43 nucleocapsid protein. Proteins were extracted from infected and treated cells after 48 hours and probed with antibodies against the HCoV-OC43 nucleocapsid protein and GAPDH as a loading control (FIG. 14). While Nilotinib-treated cells exhibited nearly complete suppression of nucleoprotein expression, TK018, TL020, TL027, and VGD020 significantly reduced nucleocapsid expression compared to the DMSO control. These results confirm the antiviral activity of these compounds and their ability to suppress viral replication and transcription, evidenced by a reduction in protein production. Plaque assays to determine the number of infectious virions accumulated in the supernatant following compound treatments were also performed. Supernatants from HCoV-OC43-infected and treated cells were collected, serially diluted (500 μL, 50 μL, and 5 μL), and used to infect monolayers of Vero-TMPRSS2 cells. After a 5-day incubation with a 1.5% CMC overlay, monolayers were fixed and stained with crystal violet to visualize plaques. Supernatant from the TK018, TL020, and VGD020 treated cells showed no visible plaques, demonstrating effective inhibition of HCoV-OC43 replication. Although the supernatant from the Nilotinib-treated cells showed no PFU/infectious virions, it may have been due to cytotoxicity rather than antiviral specificity (FIG. 15). To quantify the antiviral potency of TK018, IC50 values were determined by treating HCoV-OC43-infected cells with varying compound concentrations. After 48 hours post-treatment, the accumulation of intracellular viral genomic copies, following replication, was quantified using RT-qPCR among these treated cells. The data showed that TK018 demonstrated a dose-dependent reduction in viral RNA levels, with an IC50 value of 3.31 μM, underscoring its potent antiviral activity against HCoV-OC43 (FIGS. 16-17). Overall, TK018 emerged as a highly effective inhibitor of HCoV-OC43 replication, demonstrating potent antiviral activity with minimal cytotoxic effects. Other compounds, including TL020, TL027, and VGD020, showed significant antiviral efficacy. These findings highlight the potential of CADA compounds, particularly TK018, as promising candidates for further antiviral development

CADA Compounds were Relatively Less Potent in Inhibiting Alphacoronavirus (HCoV-229E) Replication

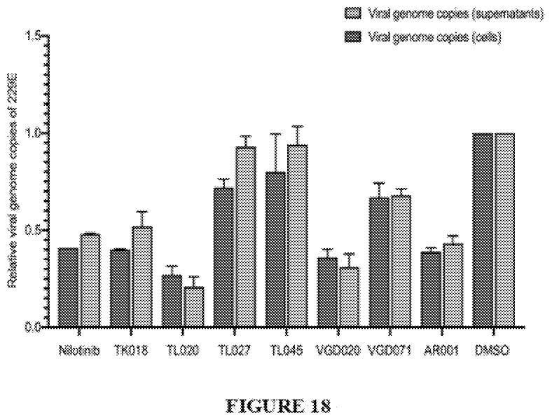

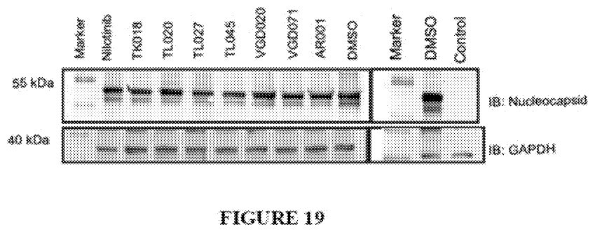

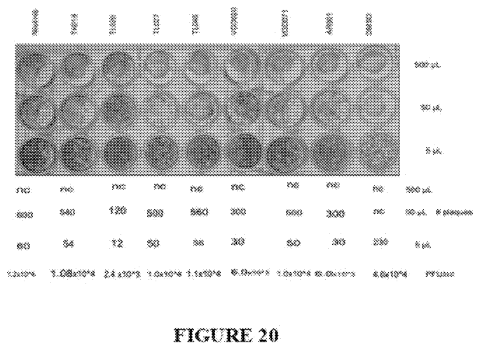

To determine the broader antiviral effects of these CADA compounds, these compounds on alpha human coronavirus-HCoV-229E were evaluated by infecting Vero-TMPRSS2 cells. Despite significant anti-viral effects against SARS-CoV-2 and HCoV-OC43, the CADA compounds exhibited relatively modest inhibition of HCoV-229E replication, as demonstrated through replication assays. First, the relative viral genome copy numbers in the supernatant and cell lysates were quantified (FIG. 18). Compounds Nilotinib, TK018, TL020, VGD020, and AR001 showed partial reductions in viral copies in both cells and the supernatant, compared to the DMSO control, which showed high viral genome copies indicative of robust replication. Conversely, TL027, TL045, and VGD071 had minimal to no effect on viral genome copies. The results suggest that while certain CADA compounds exerted some inhibitory effects, their efficacy was limited against this alphacoronavirus. Viral protein expression by immunoblotting the cell lysates of the CADA compounds and DMSO control-treated cells for HCoV-229E nucleocapsid (N) protein and GAPDH as a loading control (FIG. 19) were also assessed. The nucleocapsid protein was detected among all the tested compounds, including TK018, TL020, VGD020, and AR001, and was comparable to the DMSO-treated control. This indicates that the compounds did not significantly suppress the expression of viral structural proteins, aligning with the modest reduction in viral RNA observed in FIG. 18. The consistent detection of GAPDH confirmed equal protein loading across the samples. Finally, the impact of the compounds on the production of infectious viral particles was evaluated using a plaque assay (FIG. 20). The number of PFUs was modestly lower in the supernatant from cells treated with TL020, VGD020, and AR001, demonstrating inhibition of HCoV-229E replication. However, the PFU counts in the supernatant from cells treated with Nilotinib and TL045 were not as pronounced as those previously observed for HCoV-OC43. The supernatant from the DMSO-treated control exhibited high plaque counts, consistent with active viral replication. These findings suggest that while CADA compounds have some antiviral activity against HCoV-229E, their potency is substantially lower than against HCoV-OC43, highlighting a potential difference in their effectiveness against different coronavirus species.

The threat posed by human coronaviruses, including SARS-CoV-2, HCoV-OC43, HCoV-229E, and other related viruses, emphasizes the need to develop broad-spectrum antiviral therapies. Current antivirals have shown varying degrees of efficacy, but many are limited by their specificity to strains, susceptibility to viral mutations, or undesirable toxicities(34). This warrants the development of drugs targeting conserved viral pathways, thereby enabling robust activity across diverse coronavirus groups. The available antiviral therapies for SARS-CoV-2 have primarily centered on repurposed drugs and new agents targeting specific viral proteins or mechanisms (35). Remdesivir, an RdRp inhibitor, has been one of the most prominent antivirals during the COVID-19 pandemic (36). It acts as a nucleoside analog, incorporating into viral RNA and causing chain termination, effectively halting replication (37). Despite its efficacy in reducing recovery time in hospitalized COVID-19 patients, remdesivir's utility is limited by its intravenous mode of administration and modest impact on mortality rates (38). Another nucleoside analog, molnupiravir, introduces catastrophic mutations in the viral RNA genome, impairing its replication capability (39). While molnupiravir demonstrated significant efficacy in early clinical trials, its effectiveness diminishes in patients with high viral loads, highlighting the need for complementary therapies (34). Ribavirin, a broad-spectrum antiviral, has also been used against SARS and MERS coronaviruses, but its high toxicity and limited efficacy in vivo have restricted its widespread use (40) The protease inhibitors nirmatrelvir and ritonavir, such as Paxlovid, have emerged as another frontline therapy for SARS-CoV-2 (13). By inhibiting the 3CL protease, a critical enzyme for viral polyprotein processing, Paxlovid effectively curtails viral replication and has been shown to reduce hospitalization and death in high-risk individuals when administered early in infection (13). However, the reliance on specific protease targets renders it vulnerable to resistance from mutations in the protease gene. This study explored the potential of CADA compounds as broad-spectrum inhibitors targeting the highly conserved RdRp enzyme, a central component of the coronavirus replication machinery. TK018 emerged as a lead candidate among the tested compounds, demonstrating potent inhibition of RdRp activity in vitro with minimal cytotoxicity. The compound reduced viral RNA levels and the production of infectious viral particles in the supernatant of SARS-CoV-2 and HCoV-OC43 infected cells. TK018 exhibited an IC50 of 4.5 μM for SARS-CoV-2 and 3.31 μM for HCoV-OC43, demonstrating its strong efficacy. The ability of TK018 to target a conserved viral mechanism like RdRp makes it less susceptible to resistance from spike protein mutations, a limitation faced by monoclonal antibodies and entry inhibitors. The findings from this study align with and extend previous research on repurposing drugs like niclosamide and broad-spectrum peptides for coronavirus management (41). By targeting conserved viral mechanisms such as RdRp, TK018 reduces the likelihood of resistance arising from mutations, which is critical for long-term efficacy against emerging variants.

This study also highlighted the differential efficacy of CADA compounds against various coronavirus strains. While TK018 showed potent activity against betacoronaviruses such as SARS-CoV-2 and HCoV-OC43, its impact on the alphacoronavirus HCoV-229E was modest. This differential efficacy suggested the need for structural studies of RdRp across coronavirus genera to identify conserved features that can be exploited for broad-spectrum activity. The limited activity of TK018 against HCoV-229E highlights an opportunity for further optimization of the compound to broaden its antiviral spectrum. This study provides a significant step forward in developing an anti-coronavirus antiviral. First, it introduces CADA compounds as a new class of antivirals with demonstrated broad-spectrum activity. Second, it provides an in-vitro framework for evaluating the efficacy of potential antiviral candidates, including cell-based RdRp activity assay, quantitation of replicated viral RNA, and estimation of infectious virions in the treated cells through plaque assay. These methodologies are essential for identifying and evaluating inhibitors for targeting viral replication. This study also highlights the need to balance the efficacy of the antiviral activity and the cytotoxic effect, a delicate but essential consideration to ensure antivirals can be both efficacious and harmless.

Building on TK018's promising results, future research should focus on structural biology analyses to optimize its binding affinity for RdRp across coronavirus strains. Additionally, combination therapies, including TK018 with existing antivirals, could enhance therapeutic outcomes by exploiting complementary pathways to inhibit viral replication. Investigating the role of TK018 in inhibiting zoonotic coronaviruses, which pose a risk of future spillover events, can further validate its potential as a broad-spectrum antiviral. In conclusion, this study takes a significant step forward in antiviral research by showing how the cell-based RdRp assay can successfully identify antiviral compounds with broad-spectrum potential. Through this work, TK018 was identified as a promising candidate for further development. Importantly, this study offers valuable progress in addressing the current coronavirus outbreaks and lays the foundation for developing antivirals for future outbreaks.

All reactions were performed under an atmosphere of dry nitrogen. Reagents and solvents purchased from Aldrich Chemical Company, Acros Organics, or Fisher Scientific were of ACS reagent grade or better and were used without purification, unless indicated otherwise. Anhydrous acetonitrile (AN) was distilled from CaH₂. Anhydrous N,N-dimethylformamide (DMF) was obtained from Sigma Aldrich. Solutions of 2 N HCl in methanol/water were prepared from 42 mL of concentrated aqueous HCl (12.1 N) and 210 mL of methanol. Trituration of HCl salts was done by sonication in anhydrous diethyl ether (5-15 mL, unless indicated otherwise) for 5 min and filtration, repeating the process two more times. “Overnight” periods are ca. 16 h. Organic solutions were dried with anhydrous Na₂SO₄, and sample drying in vacuo was performed at 0.1 mm at room temperature. Column chromatography was performed with Sorbent Technologies neutral alumina (50-200 μm) or standard grade silica (32-63 μm), unless noted otherwise. Automated chromatography was performed on a Yamazen Smart Flash AKROS RE-X10 with Sorbent Technologies neutral alumina (50-200 μm) or Sorbent Technologies standard grade silica (32-63 μm). Melting points were measured on a Thomas-Hoover or Mel-Temp apparatus and are uncorrected. ¹H NMR (400 MHz or 500 MHz) and ¹³C NMR (100 MHz or 125 MHz) spectra were acquired on a Varian 400 or a Varian Unity+500 spectrometer. All chemical shifts (6) are reported in ppm units relative to solvent resonances, as follows: ¹K, CDCl₃/TMS=0.00; ¹³C, CDCl₃=77.16. Infrared spectra (IR) were recorded neat on a Nicolet 6700 FTIR spectrometer. Low-resolution mass spectra (MS) were acquired on a Waters Micromass ZQ electrospray ionization quadrupole mass spectrometer with positive ion detection (capillary voltage=3.5 kV). High-resolution mass spectra (HRMS) were determined on an Agilent 6230 TOF mass spectrometer. Samples for combustion analysis were dried at 78 °C (0.1 mm) for 2 d, unless stated otherwise, and microanalysis was performed by NuMega Resonance Labs, Inc. Samples for biological testing were at least 95% pure, as shown by combustion microanalysis.

The chemical structures of the tested compounds are shown below:

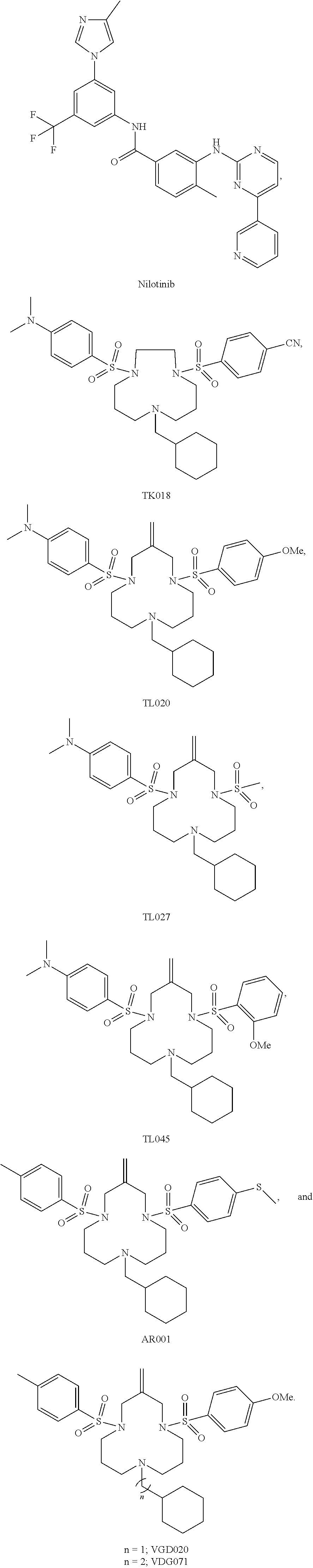

Among the above eight tested compounds, Nilotinib was bought from Sigma-Aldrich, while the other seven compounds were synthesized in Dr. Bell's labs. The synthesis of VGD020 (42, 43) VGD071 (43) and TL020 (43) has already published in the literature, while the experimental synthesis of remaining compounds is discussed below. The synthetic route for the new compounds is shown below:

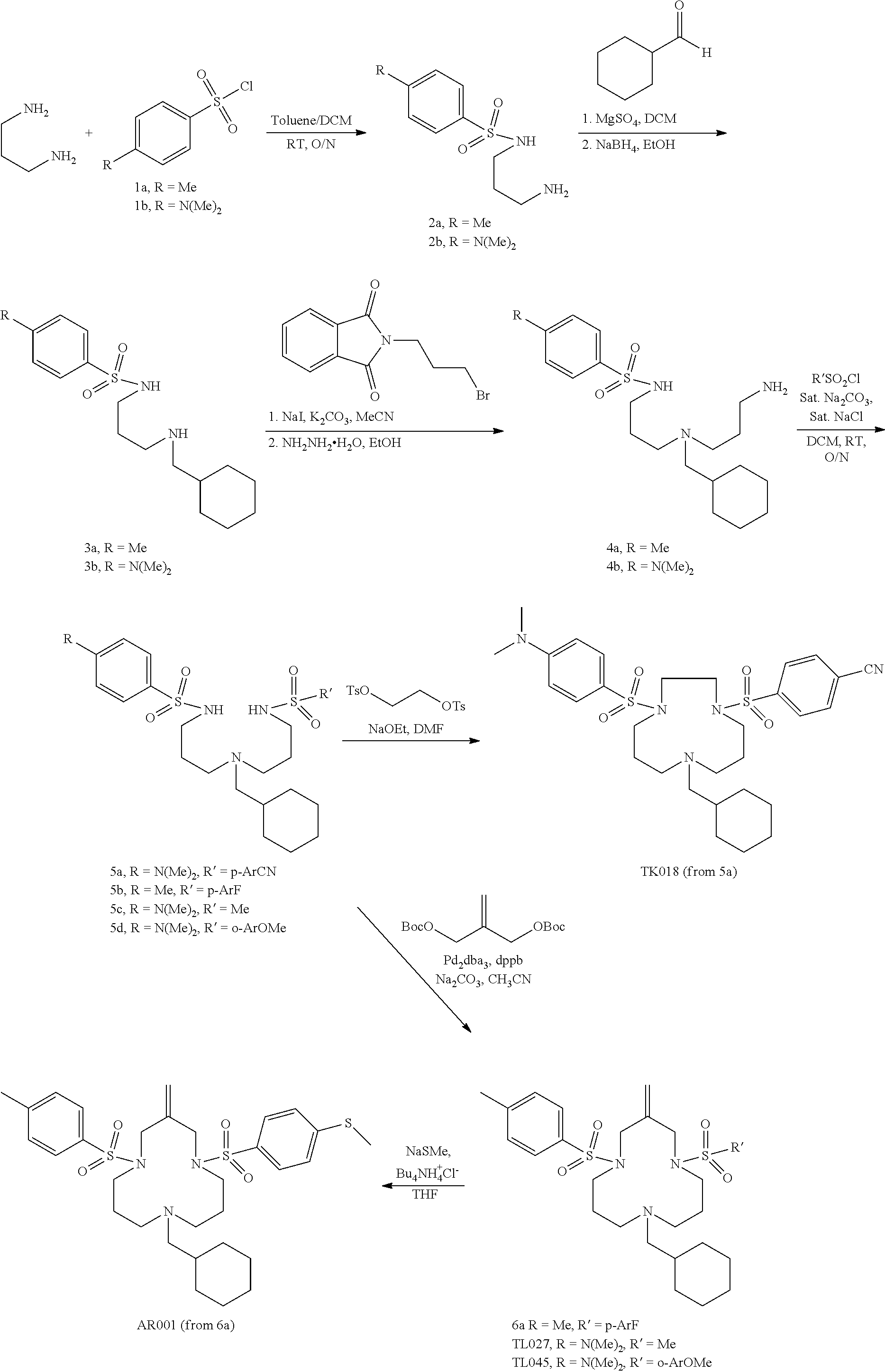

Synthesis of four unsymmetrical cyclotriazadisulfonamide CADA (42, 44) compounds were carried out starting with previously reported analogs 4a and 4b (42, 43). Synthesis of remaining compounds are shown in below.

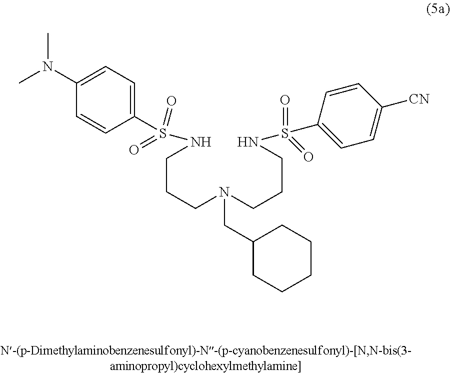 [SMILES: CN(C)c1ccc(S(=O)(=O)NCCCN(CCCNS(=O)(=O)c2ccc(C#N)cc2)CC2CCCCC2)cc1]

A mixture of 6.6 g (16 mmol) of 4b, 4.2 g (21 mmol) of p-cyanobenzenesulfonyl chloride, 150 mL of saturated aqueous NaCl solution, 150 mL of saturated aqueous Na₂CO₃ solution, and 150 mL of DCM was stirred vigorously under nitrogen at room temperature for 24 h. The layers were separated, and the aqueous layer was extracted with DCM (3×100 mL). The combined organic solutions were dried and filtered. The filtrate was concentrated by rotary evaporation, and the residue was dried in vacuo, yielding 8.96 g (97%) of a viscous yellow oil. The crude product was stirred with 120 mL of 2 M HCl in aqueous methanol for 4 h, then the solvent was removed by rotary evaporation. The residue was dried in vacuo, triturated with diethyl ether (3×50 mL), and dried again in vacuo giving 7.7 g (77%) of 5a·HCl as a light yellow solid. A small sample (ca. 10 mg) was removed and dried for microanalysis. IR (neat cm⁻¹) 2930 (w), 2853 (w), 2437 (w, br), 1675 (w), 1598 (w), 1449 (w), 1328 (m), 1160 (s), 1130 (m), 1093 (m), 995 (w), 901 (w), 840 (w), 785 (w), 656 (m), 629 (m), 573 (s). Anal. Calcd for C₂₈H₄₁N₅O₄S₂·2HCl·0.5H₂O·0.5DCM: C, 48.89; H, 6.48; N, 10.00. Found, C, 48.85; H, 6.12; N, 10.15. The HCl salt was converted back to the free base by stirring vigorously with 100 mL of 2 M aqueous NaOH solution, 100 mL of saturated aqueous NaCl solution, and 100 mL of DCM for 4 h. The layers were separated, and the aqueous layer was extracted with DCM (3×50 mL). The combined organic solutions were dried and filtered. The filtrate was concentrated by rotary evaporation, and the residue was dried in vacuo, giving 6.80 g (73% overall) of 5a as a light-yellow viscous liquid. ¹H NMR (500 MHz, CDCl₃) δ 7.97 (d, 9 Hz, 2H, m-ArCN), 7.78 (d, 9 Hz, 2H, o-ArCN), 7.65 (d, 9 Hz, 2H, m-ArN(CH₃)₂), 6.67 (d, 9 Hz, 2H, o-ArN(CH₃)₂), 3.04 (s, 8H, N(CH₃)₂, CH₂NHSO₂ArCN), 2.93 (t, 6 Hz, 2H, CH₂NHSO₂ArN(CH₃)), 2.37 (m, 4H, CH₂NCH₂Cy), 2.04 (d, 7 Hz, 2H, CH₂Cy), 1.62 (m, 10H, CCH₂C, Cy), 1.14 (m, 3H, Cy), 0.78 (m, 2H, Cy). ¹³C NMR (125 MHz, CDCl₃) δ 152.8, 144.7, 132.9, 128.9, 127.7, 124.7, 117.5, 115.9, 110.9, 62.1, 53.2, 52.9, 42.8, 42.4, 40.1, 35.6, 31.9, 26.6, 26.0, 25.8. HRMS calcd for C₂₈H₄₁N₅O₄S₂ (MH)⁺ 576.2673; Found 576.2686 (M+H)⁺.

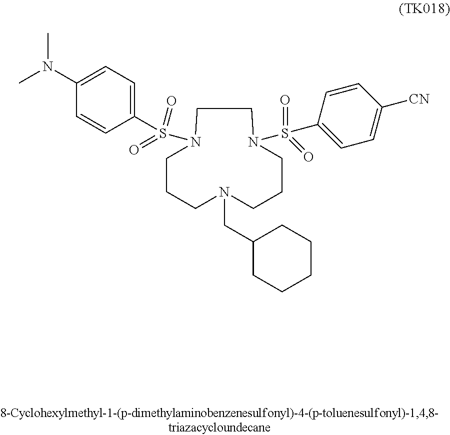 [SMILES: CN(C)c1ccc(S(=O)(=O)N2CCCN(CC3CCCCC3)CCCN(S(=O)(=O)c3ccc(C#N)cc3)CC2)cc1]

In an oven-dried apparatus, a solution of 6.75 g (11.7 mmol) of 5a in 50 mL anhydrous ethanol was boiled at reflux under nitrogen and 18 mL of a 2 M solution of sodium ethoxide in ethanol was added. The resulting solution was cooled to room temperature and solvent was removed under vacuum under anhydrous conditions. A solution of the resulting disodium salt in 25 mL of anhydrous DMF was stirred at 110 °C under nitrogen and a solution of 4.8 g (13 mmol) of ethylene glycol ditosylate (4) in 25 mL of anhydrous DMF was added slowly over 12 h by means of a syringe pump. The reaction mixture was stirred for 12 h at 110 °C, then cooled to room temperature, diluted with 500 mL of water, and extracted with DCM (3×50 mL). The combined organic layers were dried and filtered. The filtrate was concentrated to dryness by rotatory evaporation, and the residue was purified by column chromatography on silica, eluting with hexane/ethyl acetate/triethylamine (70:30:1, v/v/v), yielding 0.88 g (12%) of TK018 as a colorless glassy solid. ¹H NMR (400 MHz, CDCl₃) δ 7.93 (d, 9 Hz, 2H, m-ArCN), 7.83 (d, 9 Hz, 2H, o-ArCN), 7.60 (d, 9 Hz, 2H, m-ArN(CH₃)₂), 6.68 (d, 9 Hz, 2H, o-ArN(CH₃)₂), 3.49 (m, 2H, NCH₂CH₂N), 3.26 (m, 4H, NCH₂CH₂N, CH₂NHSO₂ArCN), 3.05 (s, 6H, N(CH₃)₂), 3.03 (m, 2H, CH₂NHSO₂ArN(CH₃)₂), 2.53 (m, 4H, CH₂NCH₂Cy), 2.09 (d, 7 Hz, 2H, CH₂Cy), 1.66 (m, 10H, CCH₂C, Cy), 1.17 (m, 3H, Cy), 0.78 (m, 2H, Cy). ¹³C NMR (100 MHz, CDCl₃) δ 152.9, 142.5, 132.9, 129.4, 128.1, 122.1, 117.3, 116.4, 110.9, 62.5, 51.0, 50.5, 50.0, 49.7, 48.9, 48.1, 40.0, 35.6, 32.2, 26.7, 26.1, 24.1, 23.4. HRMS calcd for C₃₀H₄₃N₅O₄S₂ (MH)⁺ 602.2848; Found 602.2829 (M+H)⁺. 0.2 g sample of the free base was converted to HCl salt by stirring with 25 mL of 2 M HCl in aqueous methanol for 4 h, then the solvent was removed by rotary evaporation. The residue was dried in vacuo, triturated with anhydrous diethyl ether (3×25 mL), then dried again in vacuo, giving 0.19 g (91%) of TK018·HCl as a white solid, mp 165-167 °C. IR (neat, cm⁻¹) 2929 (w), 2855 (w), 2232 (w, br), 1596 (m), 1519 (w), 1483 (w), 1450 (w), 1336 (s), 1232 (w), 1153 (s), 1090 (m), 1015 (w), 964 (w), 943 (w), 903 (w), 841 (w), 787 (w), 774 (w), 721 (w), 683 (s), 642 (w), 618 (m), 567 (m), 548 (m), 535 (w). Anal. Calcd for C₃₀H₄₃N₅O₄S₂·2HCl·1.5H₂O: C, 51.35; H, 6.89; N, 9.98. Found, C, 51.48; H, 6.64; N, 9.94.

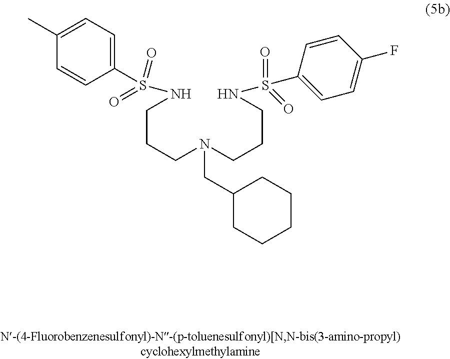 [SMILES: Cc1ccc(S(=O)(=O)NCCCN(CCCNS(=O)(=O)c2ccc(F)cc2)CC2CCCCC2)cc1]

Into a 100-mL round bottomed flask equipped with a nitrogen inlet and a magnetic stir bar, 3.73 g (9.78 mmol) of 4a, 1.91 g (9.7 mmol) of 4-fluorobenzenesulfonyl chloride, 100 mL of DCM, 100 mL of sat. aq. Na₂CO₃ and 100 mL of sat. aq. NaCl were added. The mixture was stirred vigorously at room temperature for 24 h and then transferred into a separatory funnel and the layers were separated. The aqueous layer was then extracted with DCM (2×30 mL). The combined organic extracts were dried over anhydrous Na₂SO₄, the drying agent was removed by filtration and the filtrate was concentrated by rotary 31 evaporation. The residue was further dried in vacuo (ca. 0.1 mm) yielding 6.2 g (>100%) of crude free base. The crude was purified by conversion into HCl salt and followed by trituration with anhydrous diethyl ether to remove impurities. A solution of the crude product in 40 mL of 2N HCl in MeOH was stirred at room temperature for 5 h. The solution was then concentrated by rotary evaporation and the residue was further dried in vacuo (ca. 0.1 mm) yielding 3.5 g of compound. The HCl salt was then converted into free base by stirring a solution of the compound in 60 mL of DCM, 60 mL of 2N NaOH and 60 mL of saturated (aq) NaCl for 4 h. The mixture is then poured in a separatory funnel to separate the layers and then the aqueous layer was extracted with DCM (2×15 mL). The combined organic layers were dried over anhydrous Na₂SO₄. The drying agent was removed by filter and the filtrate was concentrated by rotary evaporation and dried in vacuo (ca. 0.1 mm) yielding 3.42 g (65%) of 5b as pale-yellow oil. Freebase 1H NMR (400 MHz, CDCl₃) 1H NMR (400 MHz, CDCl₃) δ 7.94-7.78 (m, 2H), 7.79-7.62 (m, 2H), 7.38-7.27 (m, 2H), 7.24-7.05 (m, 2H), 5.88 (bs, 2H, NH), 2.99 (dt, J=10.2, 6.3 Hz, 4H), 2.53-2.23 (m, 7H), 2.04 (d, J=6.9 Hz, 2H), 1.76-1.40 (m, 10H), 1.12 (d, J=8.3 Hz, 2H), 1.17 (m, 2H), 0.79 (s, 1H). 13C NMR (126 MHz, CDCl₃) δ 166.03, 164.00, 143.43, 136.33, 135.55, 130.05, 129.98, 129.81, 127.24, 116.45, 116.28, 60.39, 51.83, 40.40, 33.37, 31.50, 25.67, 25.37, 23.43, 21.57. IR (neat, cm−1) 3276 (w), 2922 (m), 2849 (w), 1717 (w), 1654 (w), 1591 (w), 1493 (w), 1448 (w), 1405 (w), 1322 (w), 1290 (w), 1233 (w), 1150 (w), 1091 (w), 891 (w), 837 (w), 813 (w), 706 (w). MS (ESI+) m/z 540.30 (MH+). mp 97-100 °C. Anal. Calcd. for C₂₆H₃₉ClFN₃O₄S₂C, 54.20; H, 6.82; N, 7.29. Found: C, 54.50; H, 7.00; N, 7.58.

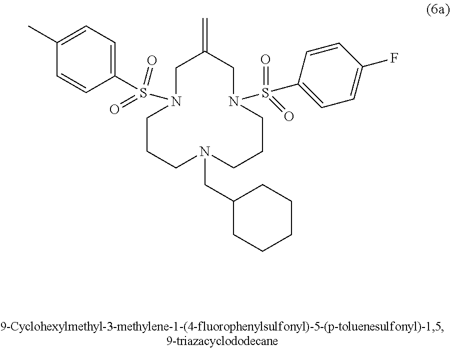 [SMILES: C=C1CN(S(=O)(=O)c2ccc(C)cc2)CCCN(CC2CCCCC2)CCCN(S(=O)(=O)c2ccc(F)cc2)C1]

Into a 250-mL round-bottomed flask equipped with a condenser, a nitrogen inlet and a magnetic stirring bar, a solution of 2.5 g (4.64 mmol) of 5b, 3.83 g (13.3 mmol) of 2-methylene-1,3-propanebis(ter-butylcarbonate) (42, 44), 0.11 g (0.24 mmol) of 1,4-bis(diphenylphosphinobutane) (dppb), 0.12 g (0.13 mmol) of tris(dibenzylideneacetone) dipalladium (0) (Pd₂(dba)₃) and 0.251 g (2.37 mmol) of Na₂CO₃ in 247 mL of anhydrous CH₃CN was added. This mixture was refluxed for 24 h and then allowed to cool room temperature. The solvent was removed by rotary evaporation. The residue was dissolved in 50 mL of DCM, washed with of saturated (aq). NaHCO₃ (2×20 mL) and saturated (aq). NaCl (2×20 mL). The organic extracts were dried over anhydrous Na₂SO₄, filtered and rotary evaporated to give 2.6 g (>100%) of sticky crude residue. Crude was purified by alumina column chromatography by eluting with 1:3 ethyl acetate and hexane to yield 0.7 g of the 6a. This product was converted as HCl salt and triturated with 40 mL of diethyl ether and dried in vacuo to yield 0.3 g (11%) of 6a. HCl as pale yellow solid. Free base 1H NMR (400 MHz, CDCl₃) δ 7.93-7.80 (m, 2H), 7.70 (dd, J=8.1, 1.8 Hz, 2H), 7.34-7.25 (m, 2H), 7.12 (t, J=8.4 Hz, 2H), 5.24 (s, 2H), 3.08-2.84 (m, 4H), 2.53-2.26 (m, 7H), 2.01 (dd, J=6.9, 2.1 Hz, 2H), 1.73-1.49 (m, 10H), 1.37-1.01 (m, 7H), 0.84-0.61 (m, 2H).13C NMR (126 MHz, CDCl₃) δ 166.45, 164.41, 144.41, 141.53, 133.37, 132.60, 132.58, 130.35, 130.28, 130.07, 127.54, 119.60, 116.86, 116.68, 61.04, 53.01, 52.43, 48.86, 48.65, 47.30, 33.67, 31.59, 25.66, 25.50, 21.56, 20.35, 20.04. IR (neat, cm−1) 2925 (w), 2852 (w), 1590 (w), 1492 (w), 1448 (w), 1333 (w), 1293 (w), 1229 (w), 1153 (w), 1087 (w), 1015 (w), 898 (w), 876 (w), 838 (w), 818 (w), 780 (W), 724 (W), 709 (w). MS (ESI+) m/z 592.32 (MH+). 33 mp 145 °C (dec 109-115 °C). Anal. Calcd. For C₃₀H₄₃ClFN₃O₄S₂. 0.5 H2O: C, 56.54; H, 6.96; N, 6.59. Found: C, 56.59; H, 7.06; N, 6.53.

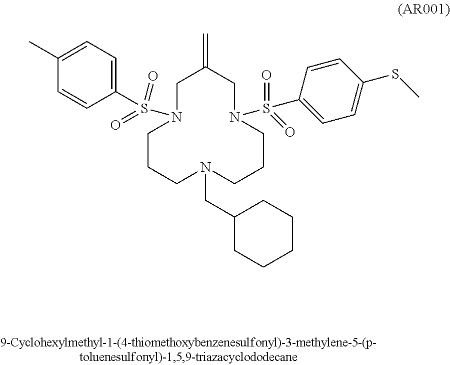

Into a round bottomed flask equipped with a nitrogen inlet and a magnetic stir bar, a solution of 0.301 g (0.51 mmol) 6a and 0.169 g (0.61 mmol) tetrabutylammonium chloride in 1 mL anhydrous THF was added under nitrogen via cannula to a stirred suspension of 0.053 g (1.06 mmol) of sodium thiomethoxide in anhydrous THF (2 mL). The mixture was stirred at room temperature for 48 h. After completion of reaction, 1 mL of methanol was added and concentrated by rotary evaporation. The residue was dissolved in 20 mL of ethyl acetate and washed with 2N NaOH (2×20 mL). The ethyl acetate layer was concentrated and dried in vacuo to yield 0.24 g of AR001 free base. AR001 free base was converted as HCl salt and triturated with 20 mL of diethyl ether, filtered, and dried in vacuo to yield 0.25 g (79%) of AR001. HCl as pale yellow solid. HCl salt 1H NMR (400 MHz, CDCl₃) δ 7.61 (d, J=7.9 Hz, 4H), 7.33 (t, J=7.9 Hz, 4H), 5.33 (s, 2H), 3.74-3.60 (m, 4H), 3.38 (s, 2H), 3.16 (m, 5H), 2.76 (s, 2H), 2.52 (s, 3H), 2.43 (s, 3H), 2.26 (d, J=15.9 Hz, 2H), 2.03-1.61 (m, 7H), 1.34-0.97 (m, 7H).13C NMR (126 MHz, CDCl₃) δ 163.42, 159.97, 146.86, 144.38, 141.80, 141.66, 133.38, 131.92, 130.05, 129.68, 127.91, 127.80, 127.60, 127.56, 125.58, 119.44, 114.61, 60.95, 52.81, 52.69, 48.71, 47.18, 34.65, 33.71, 31.60, 31.56, 29.67, 29.03, 25.67, 25.56, 25.52, 25.26, 34 24.53, 22.63, 21.58, 20.19, 14.78, 14.11, 14.01, 11.42. IR (neat, cm⁻¹) 2923 (w), 2852 (w), 1579 (w), 1448 (w), 1332 (w), 1262 (w), 1156 (w), 1101 (w), 1017 (w), 898 (w), 876 (w), 804 (w), 787 (w), 722 (w), 707 (w), 686 (w). MS (ESI+) m/z 620.76 (MH+). mp 155-165 °C. Anal. Calcd. For C₃₁H₄₆ClN₃O₄S₃: C, 56.73; H, 7.04; N, 6.40. Found: C, 57.00; H, 6.73; N, 6.06.

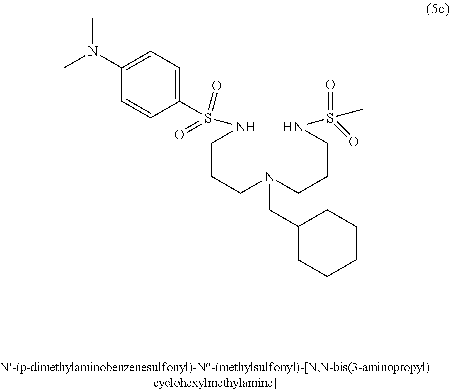

To a 250 mL round bottom flask, 1.75 g (4.26 mmol) of 4b, 0.611 g (5.33 mmol) of methane sulfonyl chloride, 15 mL of sat. aq. NaCl solution, 15 mL of sat. aq. Na₂CO₃ solution, and 15 mL of DCM were added. The mixture was stirred vigorously at room temperature for 24 h. The mixture was then placed in a separatory funnel and the organic layer was removed. The aqueous layer was extracted with DCM (3×15 mL), the combined extraction layers were dried (Na₂SO₄), and filtered. The filtrate was then concentrated, and the resulting residue was dried in vacuo. The product was converted to the HCl salt by stirring with 15 mL of a solution of 2 N HCl in MeOH and stirring for 1 h. The solution was concentrated by rotary evaporation and the resulting residue was dried in vacuo. The solids were triturated with diethyl ether (3×15 mL) and the resulting residue was dried in vacuo. The product was converted back to the free base by stirring vigorously with 15 mL of 2 N aq. NaOH solution, 15 mL of sat. aq. NaCl solution, and 15 mL of DCM for 1 h. The layers were separated, and the aqueous layer was extracted with DCM (3×15 mL). The combined organic layer was dried (Na₂SO₄) and filtered. The filtrate was concentrated by rotary evaporation and the resulting residue was dried in vacuo. This produced 1.87 g (90%) of 5c as a yellow viscous oil. 1H NMR (400 MHz, CDCl₃) δ 7.64 (m, 2H, o-ArSO₂), 6.65 (m, 2H, m-ArSO₂), 5.72 (bs, 2H, NH), 3.16 (t, 6.4 Hz, 4H, CH₂N), 3.01 (s, 6H, N(CH₃)₂), 2.92 (s, 3H, CH₃), 2.39 (dt, 13.2 Hz, 6.3 Hz, 4H, CH₂N), 2.06 (d, 6.9 Hz, 2H, CH₂Cy), 1.64 (m, 10H, CCH₂C, Cy), 1.37 (m, 1H, CH) 1.16 (m, 2H, Cy), 0.80 (m, 2H, Cy). 13C NMR (101 MHz, CDCl₃) δ 152.7, 124.9, 111.0, 110.9, 62.0, 53.1, 42.6, 42.5, 40.1, 39.7, 35.7, 31.9, 26.6, 26.5, 26.0, 25.8. IR (neat cm⁻¹) 2926 (m), 2851 (m), 2497 (w), 2436 (w), 1596 (w), 1448 (m), 1312 (s), 1149 (s), 1093 (s), 975 (m), 900 (w), 842 (m), 770 (m), 655 (s). MS (ESI+) m/z 489 (MH+). Anal. Calcd for C₂₂H₄₀N₄O₄S₂HClH₂O: C, 48.65; H, 7.98; N, 10.31. Found: C, 48.76; H, 8.08; N, 10.03.

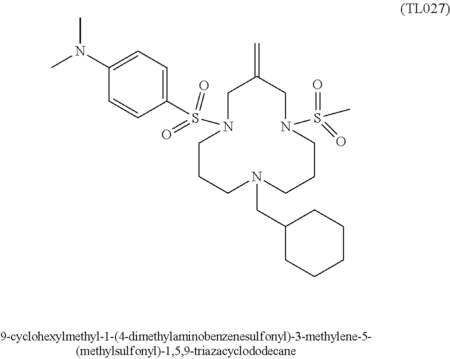

To a 500 mL round bottom flask, 0.957 g (1.96 mmol) of 5c, 2.56 g (8.88 mmol) of 2-methylene-1,3-propanebis(tert-butylcarbonate), 0.243 g (2.29 mmol) of Na₂CO₃, 0.108 g (0.118 mmol) of Pd₂dba₃, 0.110 g (0.258 mmol) of dppb and 218 mL of anhydrous acetonitrile were stirred under N2 gas and boiled under reflux. The mixture was then allowed to cool to room temperature and filtered. The filtrate was washed with 50 mL of sat. aq. NaHCO₃ solution. The organic layer was separated, and the aqueous layer was extracted with DCM (3×25 mL). The combined organic solutions were dried (Na₂SO₄) and filtered. The filtrate was concentrated by rotary evaporation and the resulting residue was dried in vacuo. The product was converted to the HCl salt by stirring with 25 mL of a solution of 2 N HCl in MeOH for 1 h. The solution was then concentrated by rotary evaporation and the resulting residue was dried in vacuo. The resulting solid was triturated with diethyl ether (3×25 mL) and the residue dried in vacuo. The product was then converted back to the free base by stirring vigorously with 25 mL of DCM, 25 mL of aq. 2 N NaOH solution, and 25 mL sat. aq. NaCl solution for 4 h. 148 The layers were separated, and the aqueous layer was extracted with DCM (3×25 mL). The combined organic solutions were dried (Na₂SO₄) and filtered. The filtrate was concentrated, and the resulting residue was dried in vacuo. The product was purified by automated chromatography on neutral alumina, eluting with 1:1 (v/v) ethyl acetate/hexane. This produced 344 mg (33%) of TL027 as a yellow viscous oil. 1H NMR (400 MHz, CDCl₃) δ 7.59 (m, 2H, o-ArSO₂), 6.69 (m, 2H, m-ArSO₂), 5.21 (d, 37.9 Hz, 2H, C═CH₂), 3.99 (s, 2H, NCH₂), 3.60 (s, 2H, NCH₂), 3.43 (t, 7.6 Hz, 2H, NCH₂), 3.06 (s, 6H, N(CH₃)₂), 2.95 (m, 2H, NCH₂), 2.88 (s, 3H, SO₂CH₃), 2.41 (t, 5.8 Hz, 2H, NCH₂), 2.30 (t, 5.8 Hz, 2H, NCH₂), 2.00 (d, 6.9 Hz, 2H, CH₂Cy), 1.67 (m, 10H, CCH₂C, Cy), 1.49 (m, 1H, Cy), 1.20 (m, 2H, Cy), 0.71 (q, 11.8 Hz, 2H, Cy). 13C NMR (101 MHz, CDCl₃) δ 152.8, 138.3, 129.2, 122.2, 116.1, 110.9, 77.3, 77.0, 76.7, 62.1, 54.1, 51.0, 49.5, 47.4, 45.6, 42.7, 40, 39.0, 35.9, 31.9, 27.7, 26.8, 26.0, 25.9, 23.5. IR (neat cm−1) 2925 (w), 2847 (w), 2416 (w), 2283 (w), 1656 (w), 1595 (w), 1516 (w), 1449 (w), 1373 (w), 1320 (w), 1225 (w), 1146 (m), 1090 (m), 1016 (w), 966 (m), 898 (w), 877 (w), 791 (m), 756 (w), 733 (w), 707 (w), 686 (w). MS (ESI+) m/z 541 (MH+), 542 (MH++1), Anal. Calcd. for C₂₆H₄₄N₄O₄S₂·HCl·0.25H₂O: C, 53.68; H, 7.88; N, 9.63. Found: C, 53.43; H,7.51; N, 9.36.

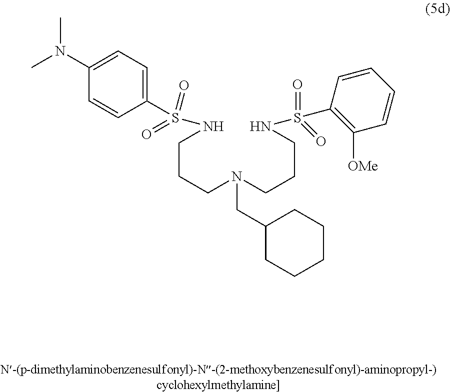 [SMILES: COc1ccccc1S(=O)(=O)NCCCN(CCCNS(=O)(=O)c1ccc(N(C)C)cc1)CC1CCCCC1]

To a 250 mL round bottom flask, 1.72 g (4.19 mmol) of 4b, 1.52 g (7.36 mmol) of 2-methoxybenzene sulfonyl chloride, 15 mL of sat. aq. NaCl solution, 15 mL of sat. aq. Na₂CO₃ solution, and 15 mL of DCM were added. The mixture was stirred vigorously at room temperature for 24 h. The mixture was then placed in a separatory funnel and the organic layer was removed. The aqueous layer was extracted with DCM (3×15 mL), the combined extraction layers were dried (Na₂SO₄), and filtered. The filtrate was then concentrated, and the resulting residue was dried in vacuo. The product was converted to the HCl salt by stirring with 15 mL of a solution of 2 N HCl in MeOH and stirring for 1 h. The solution was concentrated by rotary evaporation and the resulting residue was dried in vacuo. The solids were triturated with diethyl ether (3×15 mL) and the resulting residue was dried in vacuo. The product was converted back to the free base by stirring vigorously with 15 mL of 2 N aq. NaOH solution, 15 mL of sat. aq. NaCl solution, and 15 mL of DCM for 1 h. The layers were separated, and the aqueous layer was extracted with DCM (3×15 mL). The combined organic layer was dried (Na₂SO₄) and filtered. The filtrate was concentrated by rotary evaporation and the resulting residue was dried in vacuo. This produced 2.43 g (99%) of 5d as a yellow viscous oil. 1H NMR (400 MHz, CDCl₃) δ 7.82 (t, 6.9 Hz, 1H, m-OMePh), 7.60 (d, 8.5 Hz, 2H, o-ArSO₂), 7.48 (t, 7.9 Hz, 1H, m-OMePh), 7.21 (m, 2H, o/p-OMePh), 6.60 (d, 8.6 Hz, 2H, m-ArSO₂), 5.57 (bs, 2H, NHSO₂), 3.92 (s, 3H, OCH₃), 2.97 (s, 6H, N(CH₃)), 2.84 (m, 4H, CH₂N), 2.25 (t, 6.2 Hz, 4H, CH₂N), 1.95 (d, 6.9 Hz, 2H, CH₂Cy), 1.52 (m, 8H, CH₂C, Cy), 1.27 (m, 1H, CH), 1.09 (m, 4H, Cy), 0.69 (m, 2H, Cy). 13C NMR (101 MHz, CDCl₃) δ 156.3, 152.6, 134.4, 130.2, 128.8, 127.3, 125.1, 120.4, 112.2, 110.8, 61.7, 56.4, 53.6, 51.7, 42.8, 41.9, 40.0, 35.7, 31.8, 26.7, 26.6, 26.0, 25.6. IR (neat cm−1) 2925 (w), 2851 (w), 2601 (w), 1594 (m), 1516 (w), 1480 (m), 1434 (m), 1368 (w), 1317 (m), 1279 (m), 1248 (w), 1152 (s), 1093 (m), 1069 (m), 1015 (m), 943 (w), 899 (w), 802 (m), 758 (m), 700 (m), 646 (m), 621 (m), 614 (m). MS (ESI+) m/z 581 (MH+), 582 (MH++1). Anal. Calcd for C₂₈H₄₄N₄O₅S₂·HCl·CH₃OH: C, 53.65; H, 7.61; N, 8.63. Found: C, 53.40; H, 7.23; N, 8.34.

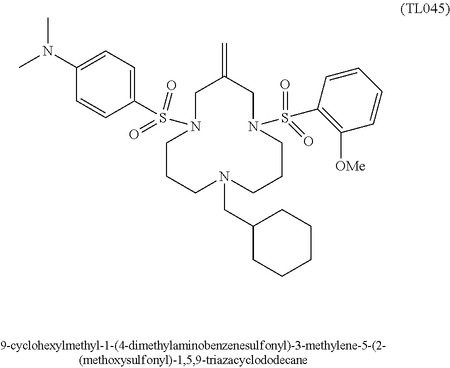 [SMILES: C=C1CN(S(=O)(=O)c2ccc(N(C)C)cc2)CCCN(CC2CCCCC2)CCCN(S(=O)(=O)c2ccccc2OC)C1]

To a 500 mL round bottom flask, 1.82 g (3.13 mmol) of 5d, 4.58 g (15.9 mmol) of 2-methylene-1,3-propanebis(tert-butylcarbonate), 179 mg (1.68 mmol) of Na₂CO₃, 173 mg (0.189 mmol) of Pd₂dba₃, 170 mg (0.399 mmol) of dppb and 348 mL of anhydrous acetonitrile were stirred under N2 gas and boiled under reflux. The mixture was then allowed to cool to room temperature and filtered. The filtrate was washed with 50 mL of sat. aq. NaHCO₃ solution. The organic layer was separated, and the aqueous layer was extracted with DCM (3×25 mL). The combined organic solutions were dried (Na₂SO₄) and filtered. The filtrate was concentrated by rotary evaporation and the resulting residue was dried in vacuo. The product was converted to the HCl salt by stirring with 25 mL of a solution of 2 N HCl in MeOH for 1 h. The solution was then concentrated by rotary evaporation and the resulting residue was dried in vacuo. The resulting solid was triturated with diethyl ether (3×25 mL) and the residue dried in vacuo. The product was then converted back to the free base by stirring vigorously with 25 mL of DCM, 25 mL of aq. 2 N NaOH solution, and 25 mL sat. aq. NaCl solution for 4 h. The layers were separated, and the aqueous layer was extracted with DCM (3×25 mL). The combined organic solutions were dried (Na₂SO₄) and filtered. The filtrate was concentrated, and the resulting residue was dried in vacuo. The product was purified by automated chromatography on neutral alumina, eluting with 3:7 (v/v) ethyl acetate/hexane. This produced 831 mg (42%) of TL045 as a clear viscous oil. 1H NMR (400 MHz, CDCl₃) δ 8.09 (d, 7.2 Hz, 1H, m-OMePh), 7.73 (m, 2H, o-ArSO₂), 7.64 (m, 1H, m-OMePh), 7.15 (m, 2H, o/p-OMePh), 6.83 (m, 2H, m-ArSO₂), 5.36 (s, 1H, C═CH₂), 5.25 (s, 1H, C═CH₂), 4.36 (s, 2H, NCH₂), 4.13 (s, 3H, OCH₃), 3.77 (s, 2H, NCH₂), 3.50 (t, 7.3 Hz, 2H, NCH₂), 3.19 (s, 6H, N(CH₃)), 3.13 (m, 2H, NCH₂), 2.49 (m, 2H, NCH₂), 2.29 (m, 2H, NCH₂), 2.09 (d, 2H, CH₂Cy), 1.81 (m, 8H, CCH₂C, Cy), 1.48 (m, 2H, Cy), 1.27 (m, 3H, Cy), 0.83 (m, 2H, Cy). 13C NMR (101 MHz, CDCl₃) δ 156.7, 152.8, 134.3, 131.1, 129.0, 120.1, 112.0, 110.9, 62.1, 60.3, 56.3, 55.9, 53.8, 50.8, 49.4, 45.7, 42.5, 40.0, 35.8, 31.8, 27.7, 26.7, 26.0, 25.9, 22.6, 21.0, 14.2. IR (neat cm⁻¹) 2924 (w), 2851 (w), 2363 (w), 1594 (m), 1516 (w), 1480 (m), 1448 (m), 1370 (w), 1320 (m), 1279 (m), 1249 (w), 1150 (s), 1091 (m), 1065 (m), 1043 (w), 1013 (m), 899 (m), 839 (w), 803 (m), 760 (m), 733 (m), 691 (m), 642 (m). MS (ESI+) m/z 633 (M+), 634 (MH+), 635 (MH++1). Anal. Calcd for C₃₂H₄₈N₄O₅S₂·HCl·H₂O: C, 55.92; H, 7.48; N, 8.15. Found: C, 56.12; H, 7.69; N, 8.10.

One of ordinary skill in the art will readily appreciate that alternative but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skills in the art to employ the present invention.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. It should also be appreciated that the numerical limits may be the values from the examples. Certain lower limits, upper limits and ranges appear in at least one claim below. All numerical values are “about” or “approximately” the indicated value, and consider experimental error and variations that would be expected by a person having ordinary skill in the art.

### REFERENCES

1. Hillen H S, Kokic G, Farnung L, Dienemann C, Tegunov D, Cramer P. 2020. Structure of replicating SARS-CoV-2 polymerase. Nature 584:154-156.
2. Jackson C B, Farzan M, Chen B, Choe H. 2022. Mechanisms of SARS-CoV-2 entry into cells. Nat Rev Mol Cell Biol. Nature Research https://doi.org/10.1038/s41580-021-00418-x.
3. Gordon D E, Jang G M, Bouhaddou M, Xu J, Obernier K, White K M, O'Meara M J, Rezelj V V., Guo J Z, Swaney D L, Tummino T A, Huttenhain R, Kaake R M, Richards A L, Tutuncuoglu B, Foussard H, Batra J, Haas K, Modak M, Kim M, Haas P, Polacco B J, Braberg H, Fabius J M, Eckhardt M, Soucheray M, Bennett M J, Cakir M, McGregor M J, Li Q, Meyer B, Roesch F, Vallet T, Mac Kain A, Miorin L, Moreno E, Naing Z Z C, Zhou Y, Peng S, Shi Y, Zhang Z, Shen W, Kirby I T, Melnyk J E, Chorba J S, Lou K, Dai S A, Barrio-Hernandez I, Memon D, Hernandez-Armenta C, Lyu J, Mathy C J P, Perica T, Pilla K B, Ganesan S J, Saltzberg D J, Rakesh R, Liu X, Rosenthal S B, Calviello L, Venkataramanan S, Liboy-Lugo J, Lin Y, Huang X P, Liu Y F, Wankowicz S A, Bohn M, Safari M, Ugur F S, Koh C, Savar N S, Tran Q D, Shengjuler D, Fletcher S J, O'Neal M C, Cai Y, Chang J C J, Broadhurst D J, Klippsten S, Sharp P P, Wenzell N A, Kuzuoglu-Ozturk D, Wang H Y, Trenker R, Young J M, Cavero D A, Hiatt J, Roth T L, Rathore U, Subramanian A, Noack J, Hubert M, Stroud R M, Frankel A D, Rosenberg O S, Verba K A, Agard D A, Ott M, Emerman M, Jura N, von Zastrow M, Verdin E, Ashworth A, Schwartz O, d'Enfert C, Mukherjee S, Jacobson M, Malik H S, Fujimori D G, Ideker T, Craik C S, Floor S N, Fraser J S, Gross J D, Sali A, Roth B L, Ruggero D, Taunton J, Kortemme T, Beltrao P, Vignuzzi M, Garcia-Sastre A, Shokat K M, Shoichet B K, Krogan N J. 2020. A SARS-CoV-2 protein interaction map reveals targets for drug repurposing. Nature 583:459-468.
4. Tian L, Qiang T, Liang C, Ren X, Jia M, Zhang J, Li J, Wan M, YuWen X, Li H, Cao W, Liu H. 2021. RNA-dependent RNA polymerase (RdRp) inhibitors: The current landscape and repurposing for the COVID-19 pandemic. Eur J Med Chem. Elsevier Masson s.r.l. https://doi.org/10.1016/j.ejmech.2021.113201.
5. Mariano G, Farthing R J, Lale-Farjat S L M, Bergeron J R C. 2020. Structural Characterization of SARS-CoV-2: Where We Are, and Where We Need to Be. Front Mol Biosci. Frontiers Media S.A. https://doi.org/10.3389/fmolb.2020.605236.
6. Zandi K, Amblard F, Musall K, Downs-Bowen J, Kleinbard R, Oo A, Cao D, Liang B, Russell O O, Mcbrayer T, Bassit L, Kim B, Schinazi R F. 2020. Repurposing Nucleoside Analogs for Human Coronaviruses.
7. Singh D D, Parveen A, Yadav D K. 2021. SARS-CoV-2: Emergence of New Variants and Effectiveness of Vaccines. Front Cell Infect Microbiol. Frontiers Media S.A. https://doi.org/10.3389/fcimb.2021.777212.
8. Liu D X, Liang J Q, Fung T S. 2020. Human Coronavirus-229E, -OC43, -NL63, and -HKU1 (Coronaviridae), p. 428-440. In Encyclopedia of Virology: Volume 1-5, Fourth Edition. Elsevier.
9. Yadav R, Chaudhary J K, Jain N, Chaudhary P K, Khanra S, Dhamija P, Sharma A, Kumar A, Handu S. 2021. cells Role of Structural and Non-Structural Proteins and Therapeutic Targets of SARS-CoV-2 for COVID-19 10:821.
10. Shirato K, Kanou K, Kawase M, Matsuyama S. 2017. Clinical Isolates of Human Coronavirus 229E Bypass the Endosome for Cell Entry. J Virol 91.
11. Metzler M, Tharyan R G, Klann K, Grikscheit K, Bojkova D, Cinatl J, Tascher G, Ciesek S, Munch C. 2023. SARS-CoV-2 Variants Show Different Host Cell Proteome Profiles With Delayed Immune Response Activation in Omicron-Infected Cells. Molecular and Cellular Proteomics 63.
12. Sabbatucci M, Vitiello A, Clemente S, Zovi A, Boccellino M, Ferrara F, Cimmino C, Langella R, Ponzo A, Stefanelli P, Rezza G. 2023. Omicron variant evolution on vaccines and monoclonal antibodies. Inflammopharmacology. Springer Science and Business Media Deutschland GmbH https://doi.org/10.1007/s10787-023-01253-6.
13. Hashemian S M R, Sheida A, Taghizadieh M, Memar M Y, Hamblin M R, Bannazadeh Baghi H, Sadri Nahand J, Asemi Z, Mirzaei H. 2023. Paxlovid (Nirmatrelvir/Ritonavir): A new approach to Covid-19 therapy? Biomedicine and Pharmacotherapy. Elsevier Masson s.r.l. https://doi.org/10.1016/j.biopha.2023.114367.
14. Tu Y F, Chien C S, Yarmishyn A A, Lin Y Y, Luo Y H, Lin Y T, Lai W Y, Yang D M, Chou S J, Yang Y P, Wang M L, Chiou S H. 2020. A review of sars-cov-2 and the ongoing clinical trials. Int J Mol Sci. MDPI A G https://doi.org/10.3390/ijms21072657.
15. Kokic G, Hillen H S, Tegunov D, Dienemann C, Seitz F, Schmitzova J, Farnung L, Siewert A, Hobartner C, Cramer P. 2021. Mechanism of SARS-CoV-2 polymerase stalling by remdesivir. Nat Commun 12.
16. Pruijssers A J, George A S, Schäfer A, Leist S R, Gralinksi L E, Dinnon K H, Yount B L, Agostini M L, Stevens L J, Chappell J D, Lu X, Hughes T M, Gully K, Martinez D R, Brown A J, Graham R L, Perry J K, Du Pont V, Pitts J, Ma B, Babusis D, Murakami E, Feng J Y, Bilello J P, Porter D P, Cihlar T, Baric R S, Denison M R, Sheahan T P. 2020. Remdesivir Inhibits SARS-CoV-2 in Human Lung Cells and Chimeric SARS-CoV Expressing the SARS-CoV-2 RNA Polymerase in Mice. Cell Rep 32.
17. Li P, Wang Y, Lavrijsen M, Lamers M M, de Vries A C, Rottier R J, Bruno M J, Peppelenbosch M P, Haagmans B L, Pan Q. 2022. SARS-CoV-2 Omicron variant is highly sensitive to molnupiravir, nirmatrelvir, and the combination. Cell Res. Springer Nature https://doi.org/10.1038/s41422-022-00618-w.
18. Kabinger F, Stiller C, Schmitzovi J, Dienemann C, Kokic G, Hillen H S, Hobartner C, Cramer P. 2021. Mechanism of molnupiravir-induced SARS-CoV-2 mutagenesis. Nat Struct Mol Biol 28:740-746.
19. Jeong J H, Chokkakula S, Min S C, Kim B K, Choi W S, Oh S, Yun Y S, Kang D H, Lee O J, Kim E G, Choi J H, Lee J Y, Choi Y K, Baek Y H, Song M S. 2022. Combination therapy with nirmatrelvir and molnupiravir improves the survival of SARS-CoV-2 infected mice. Antiviral Res 208.
20. Rosales R, Mcgovern B L, Luis Rodriguez M, Rai D K, Cardin R D, Anderson A S, Sordillo E M, Van Bakel H, Simon V, Garcia-Sastre A, White K M, Alshammary H, Banu R, Farrugia K, Silvia Gonzalez-Reiche A, Paniz-Mondolfi A, Polanco J. Nirmatrelvir, Molnupiravir, and Remdesivir maintain potent in vitro activity against the SARS-CoV-2 Omicron variant https://doi.org/10.1101/2022.01.17.476685.
21. Vangeel L, Chiu W, De Jonghe S, Maes P, Slechten B, Raymenants J, Andre E, Leyssen P, Neyts J, Jochmans D. 2022. Remdesivir, Molnupiravir and Nirmatrelvir remain active against SARS-CoV-2 Omicron and other variants of concern. Antiviral Res 198.
22. Vermeire K, Schols D, Bell T W. 2006. Inhibitors of HIV Infection via the Cellular CD4 Receptor.
23. Vermeire K, Van Laethem K, Janssens W, Bell T W, Schols D. 2009. Human Immunodeficiency Virus Type 1 Escape from Cyclotriazadisulfonamide-Induced CD4-Targeted Entry Inhibition Is Associated with Increased Neutralizing Antibody Susceptibility. J Virol 83:9577-9583.
24. Zhan P, Pannecouque C, De Clercq E, Liu X. 2016. Anti-HIV Drug Discovery and Development: Current Innovations and Future Trends. J Med Chem. American Chemical Society https://doi.org/10.1021/acs.jmedchem.5b00497.
25. Owji H, Nezafat N, Negahdaripour M, Hajiebrahimi A, Ghasemi Y. 2018. A comprehensive review of signal peptides: Structure, roles, and applications. Eur J Cell Biol. Elsevier GmbH https://doi.org/10.1016/j.ejcb.2018.06.003.
26. Vermeire K, Bell T W, Van Puyenbroeck V, Giraut A, Noppen S, Liekens S, Schols D, Hartmann E, Kalies K U, Marsh M. 2014. Signal Peptide-Binding Drug as a Selective Inhibitor of Co-Translational Protein Translocation. PLoS Biol 12.
27. Van Puyenbroeck V, Claeys E, Schols D, Bell T W, Vermeire K. 2017. A proteomic survey indicates sortilin as a secondary substrate of the ER translocation inhibitor Cyclotriazadisulfonamide (CADA). Molecular and Cellular Proteomics. American Society for Biochemistry and Molecular Biology Inc. https://doi.org/10.1074/mcp.M116.061051.
28. Goettsch C, Kjolby M, Aikawa E. 2018. Sortilin and Its Multiple Roles in Cardiovascular and Metabolic Diseases. Arterioscler Thromb Vasc Biol. Lippincott Williams and Wilkins https://doi.org/10.1161/ATVBAHA.117.310292.
29. Goettsch C, Hutcheson J D, Aikawa M, Iwata H, Pham T, Nykjaer A, Kjolby M, Rogers M, Michel T, Shibasaki M, Hagita S, Kramann R, Rader D J, Libby P, Singh S A, Aikawa E. 2016. Sortilin mediates vascular calcification via its recruitment into extracellular vesicles. Journal of Clinical Investigation 126:1323-1336.
30. Patel A B, Tsilioni I, Leeman S E, Theoharides T C. 2016. Neurotensin stimulates sortilin and mTOR in human microglia inhibitable by methoxyluteolin, a potential therapeutic target for autism. Proc Natl Acad Sci USA 113:E7049-E7058.
31. Zhong L Y, Cayabyab F S, Tang C K, Zheng X L, Peng T H, Lv Y C. 2016. Sortilin: A novel regulator in lipid metabolism and atherogenesis. Clinica Chimica Acta. Elsevier B.V. https://doi.org/10.1016/j.cca.2016.06.013.
32. Lumangtad L A, Bell T W. 2020. The signal peptide as a new target for drug design. Bioorg Med Chem Lett. Elsevier Ltd https://doi.org/10.1016/j.bmcl.2020.127115.
33. Uppal T, Tuffo K, Khaiboullina S, Reganti S, Pandori M, Verma S C. 2022. Screening of SARS-CoV-2 antivirals through a cell-based RNA-dependent RNA polymerase (RdRp) reporter assay. Cell Insight 1.
34. Santos I de A, Grosche V R, Bergamini F R G, Sabino-Silva R, Jardim A C G. 2020. Antivirals Against Coronaviruses: Candidate Drugs for SARS-CoV-2 Treatment? Front Microbiol. Frontiers Media S.A. https://doi.org/10.3389/fmicb.2020.01818.
35. Tao K, Tzou P L, Nouhin J, Bonilla H, Jagannathan P, Shafer R W. 2021. SARS-CoV-2 Antiviral Therapy. Clin Microbiol Rev. American Society for Microbiology https://doi.org/10.1128/CMR.00109-21.
36. Gordon C J, Tchesnokov E P, Woolner E, Perry J K, Feng J Y, Porter D P, Götte M. 2020. Remdesivir is a direct-acting antiviral that inhibits RNA-dependent RNA polymerase from severe acute respiratory syndrome coronavirus 2 with high potency. Journal of Biological Chemistry. American Society for Biochemistry and Molecular Biology Inc. https://doi.org/10.1074/jbc.RA120.013679.
37. Dangerfield T L, Huang N Z, Johnson K A. 2020. Remdesivir Is Effective in Combating COVID-19 because It Is a Better Substrate than ATP for the Viral RNA-Dependent RNA Polymerase. iScience 23.
38. Amstutz A, Speich B, Mentre F, Rueegg C S, Belhadi D, Assoumou L, Burdet C, Murthy S, Dodd L E, Wang Y, Tikkinen K A O, Ader F, Hites M, Bouscambert M, Trabaud M A, Fralick M, Lee T C, Pinto R, Barratt-Due A, Lund-Johansen F, Müller F, Nevalainen O P O, Cao B, Bonnett T, Griessbach A, Taji Heravi A, Schonenberger C, Janiaud P, Werlen L, Aghlmandi S, Schandelmaier S, Yazdanpanah Y, Costagliola D, Olsen I C, Briel M. 2023. Effects of remdesivir in patients hospitalised with COVID-19: a systematic review and individual patient data meta-analysis of randomised controlled trials. Lancet Respir Med 11:453-464.
39. Tian L, Pang Z, Li M, Lou F, An X, Zhu S, Song L, Tong Y, Fan H, Fan J. 2022. Molnupiravir and Its Antiviral Activity Against COVID-19. Front Immunol. Frontiers Media S.A. https://doi.org/10.3389/fimmu.2022.855496.
40. Khalili J S, Zhu H, Mak N S A, Yan Y, Zhu Y. 2020. Novel coronavirus treatment with ribavirin: Groundwork for an evaluation concerning COVID-19. J Med Virol. John Wiley and Sons Inc. https://doi.org/10.1002/jmv.25798.
41. Singh S, Weiss A, Goodman J, Fisk M, Kulkarni S, Lu I, Gray J, Smith R, Sommer M, Cheriyan J. 2022. Niclosamide-A promising treatment for COVID-19. Br J Pharmacol. John Wiley and Sons Inc https://doi.org/10.1111/bph.15843.
42. Chawla R, Van Puyenbroeck V, Pflug N C, Sama A, Ali R, Schols D, Vermeire K, Bell T W. 2016. Tuning Side Arm Electronics in Unsymmetrical Cyclotriazadisulfonamide (CADA) Endoplasmic Reticulum (ER) Translocation Inhibitors to Improve their Human Cluster of Differentiation 4 (CD4) Receptor Down-Modulating Potencies. J Med Chem 59:2633-2647.
43. Berger K, Pauwels E, Parkinson G, Landberg G, Le T, Demillo V G, Lumangtad L A, Jones D E, Islam M A, Olsen R, Kapri T, Intasiri A, Vermeire K, Rhost S, Bell T W. 2021. Reduction of Progranulin-Induced Breast Cancer Stem Cell Propagation by Sortilin-Targeting Cyclotriazadisulfonamide (CADA) Compounds. J Med Chem 64:12865-12876.
44. Demillo V G, Goulinet-Mateo F, Kim J, Schols D, Vermeire K, Bell T W. 2011. Unsymmetrical cyclotriazadisulfonamide (CADA) compounds as human CD4 receptor down-modulating agents. J Med Chem 54:5712-5721.
45. Kopiasz J, Tomaszewski W, Ku A, Chreptowicz K, Mierzejewska J, Ciach T, Ja D. 2020. Hydrophilic Quaternary Ammonium Ionenes—Is There an Influence of Backbone Flexibility and Topology on Antibacterial Properties? Macromolecular Bioscience 20:1-11; 2000063.

What is claimed is:

1. A compound or a hydrate, solvate, geometric isomer or salt thereof, wherein the compound has a chemical structure: 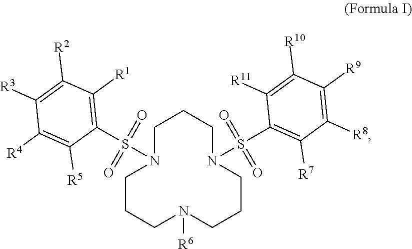
wherein R¹, R², R³, R⁴, R⁵, R⁶, R⁷, R⁸, R⁹, R¹⁰, and R¹¹ can be independently selected from the group consisting of: H; F; Cl; Br; I; OH; ketone (═O); ether [—OR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; acyl halide (—COX); carbonyl [—COR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; aldehyde (—CHO); carbonate ester [—OCOOR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; carboxyl (—COOH); amide [—CONR′R″, where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; amines [—NR′R″, where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; cyanate (—OCN); isocyanate (—NCO); nitrate (—ONO₂); nitrile (—CN); isonitrile (—NC); nitroso (—NO); oxime (—CH═NOH); borono —B(OH)₂; boronare [—B(OR′)(R″), where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; borinate [—B(R′XOR″), where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; phosophino [—PR₂, where R can include hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; phosphono [—P(═O)(OH)(R), where R can include hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; phosphate (—OP(═O)(OH)₂; thiol (—SH); sulfide [—SR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; disulfide [—SSR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers], sulfinyl [—S(═O)R, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; sulfino (—SO₂H); sulfo (—SO₃H); thiocyanate; isothiocyanate; carbonothioyl [—C(═S)R where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; (C₁₋₄)alkyl, such as methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers; and where X can be independently selected from the group consisting of: CH, S, NH, and O.

2. The compound of claim 1, wherein the compound is selected from a group consisting of:

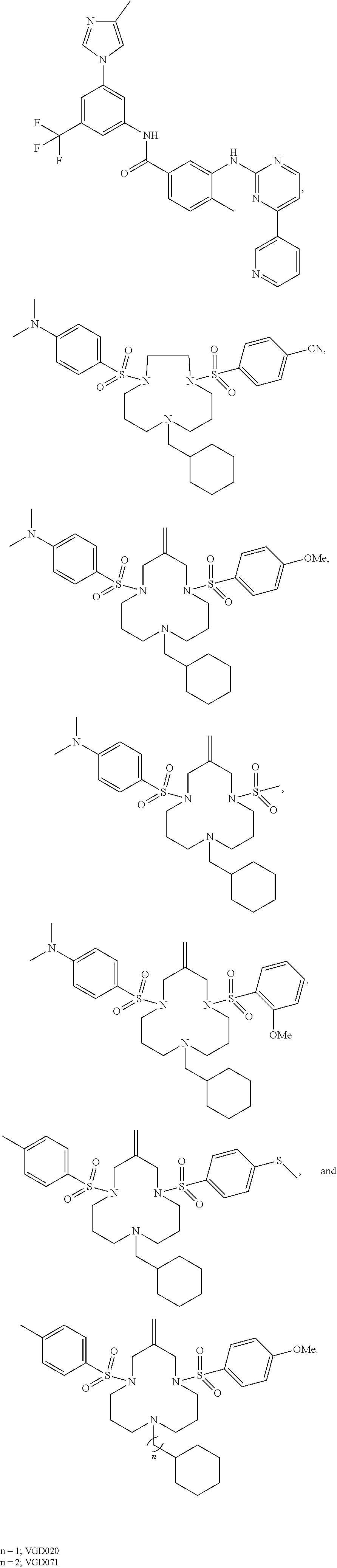

3. The compound of claim 1, wherein the compound is

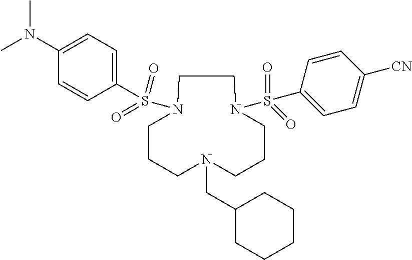 [SMILES: CN(C)c1ccc(S(=O)(=O)N2CCCN(CC3CCCCC3)CCCN(S(=O)(=O)c3ccc(C#N)cc3)CC2)cc1]

4. A pharmaceutical composition comprising a compound of claim 2 and a pharmaceutically acceptable carrier.

5. A method of treating a disease, wherein the method comprises administering to a subject a therapeutically effective amound of a compound or pharmaceutically acceptable salt of the compound with a chemical structure: 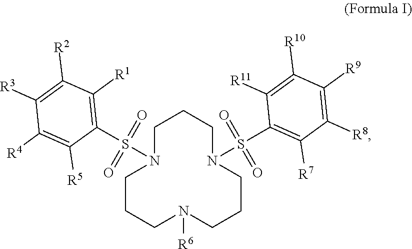
wherein R¹, R², R³, R⁴, R⁵, R⁶, R⁷, R⁸, R⁹, R¹⁰, and R¹¹ can be independently selected from the group consisting of: H; F; Cl; Br; I; OH; ketone (═O); ether [—OR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; acyl halide (—COX); carbonyl [—COR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; aldehyde (—CHO); carbonate ester [—OCOOR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; carboxyl (—COOH); amide [—CONR′R″, where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; amines [—NR′R″, where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; cyanate (—OCN); isocynate (—NCO); nitrate (—ONO₂); nitrile (—CN); isonitrile (—NC); nitroso (—NO); oxime (—CH═NOH); borono —B(OH)₂; boronare [—B(OR′)(R″), where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; borinate [—B(R′)(OR″), where R′ and R″ can be independently selected from hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; phosophino [—PR₂, where R can include hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; phosphono [—P(═O)(OH)(R), where R can include hydrogen, methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; phosphate (—OP(═O)(OH)₂; thiol (—SH); sulfide [—SR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; disulfide [—SSR, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers], sulfinyl [—S(═O)R, where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; sulfino (—SO₂H); sulfo (—SO₃H); thiocyanate; isothiocyanate; carbonothioyl [—C(═S)R where R can include methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers]; (C₁₋₄)alkyl, such as methyl, ethyl, n-propyl, isopropyl, n-butyl, sec-butyl, isobutyl, tert-butyl; (C₂₋₄)alkenyl, such as ethenyl, propenyl, butenyl, where the double bond can be located at any position in the alkenyl carbon chain, and including any alkenyl conformational isomers; and where X can be independently selected from the group consisting of: CH, S, NH, and O.

6. The method of treating a disease of claim 5, wherein the compound is selected from a group consisting of:

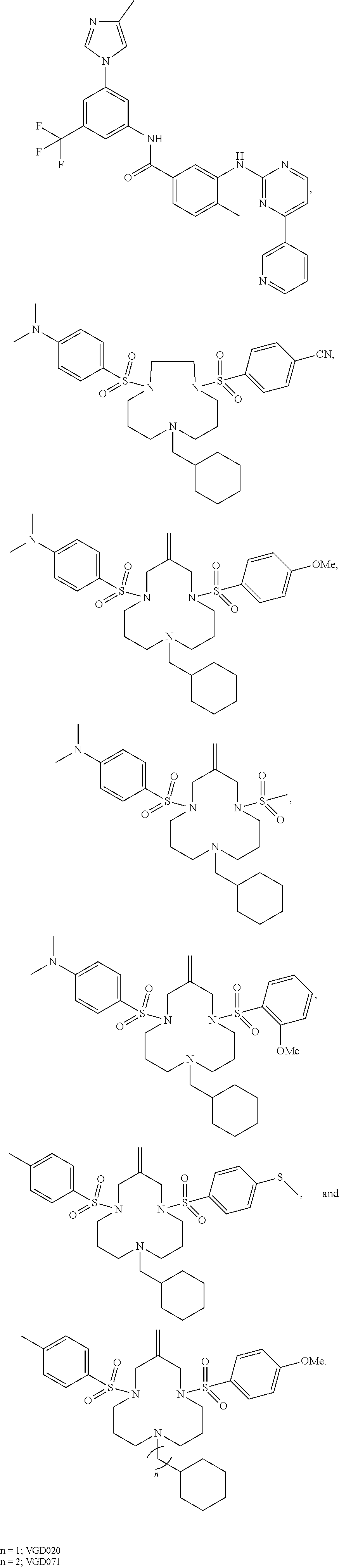

7. The method of treating a disease of claim 6, wherein the compound is 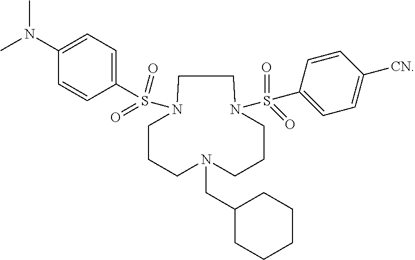 [SMILES: CN(C)c1ccc(S(=O)(=O)N2CCCN(CC3CCCCC3)CCCN(S(=O)(=O)c3ccc(C#N)cc3)CC2)cc1]
